(19) United States
(12) Patent Application Publication
(10) Pub. No.: US 2023/0233545 A1
(43) Pub. Date: Jul. 27, 2023

(54) TREATMENT OF ATRIAL DYSFUNCTION

(71) Applicant: MyoKardia, Inc., Brisbane, CA (US)

(72) Inventors: Jean-Francois TAMBY, Brisbane, CA (US); Jay M. EDELBERG, Princeton, NJ (US); Gregory Howard Takeo KURIO, Alamo, CA (US); Cynthia Lyle KELLY, San Francisco, CA (US); Chun YANG, San Mateo, CA (US); Marcus Patrick HENZE, Brisbane, CA (US); Carlos L. DEL RIO, Foster City, CA (US); Robert Lee ANDERSON, Brisbane, CA (US); Marius P. SUMANDEA, Vallejo, CA (US); Jitendra GANJU, San Francisco, CA (US)

(21) Appl. No.: 18/001,896

(22) PCT Filed: Jun. 14, 2021
(86) PCT No.: PCT/US2021/037230

§ 371 Date: Dec. 15, 2022

Related U.S. Application Data
(60) Provisional application No. 63/039,438, filed on Jun. 15, 2020.
(60) Provisional application No. 63/042,512, filed on Jun. 22, 2020.

(51) Int. Cl. A61K 31/454; A61K 31/7048; A61K 31/343; A61K 45/06; A61P 9/06

(52) U.S. Cl. CPC A61K 31/454; A61K 31/7048; A61K 31/343; A61K 45/06; A61P 9/06

(57) ABSTRACT

Provided herein are methods, uses, and compositions for treating AF in a patient, such as a patient exhibiting heart failure with reduced ejection fraction.

### CROSS-REFERENCE TO RELATED APPLICATION

This application claims priority from U.S. Provisional Patent Application 63/039,438, filed Jun. 15, 2020, and U.S. Provisional Patent Application 63/042,512, filed Jun. 22, 2020. The disclosures of those priority applications are incorporated by reference herein in their entirety.

### BACKGROUND OF THE INVENTION

Atrial fibrillation (AF) is the most common type of cardiac arrhythmia, affecting more than 37 million people worldwide. As the global population ages, the prevalence of AF is expected to increase. Patients with AF are at increased risk for stroke, cognitive decline, and cardiovascular events and mortality. AF is associated with underlying disorders such as hypertension, coronary heart disease, rheumatic heart disease, heart failure, obesity, diabetes mellitus, and chronic kidney disease. Symptoms include, but are not limited to, heart palpitations, tachycardia, shortness of breath, weakness, dizziness, fatigue, chest pain, and confusion.

AF is defined as a supraventricular tachyarrhythmia with uncoordinated atrial activation leading to ineffective atrial contraction, and can result from structural and/or electrical abnormalities of the atrium. Electrocardiographic characteristics include irregular R-R intervals (when AV conduction is present), no distinct repeating P waves, and irregular atrial activity. Episodes often increase in frequency and duration over time and become less responsive to medication. There are generally four types of AF (January et al., JACC (2014) 64(21):2246-80; Kirchhof et al., Eur Heart J. (2016) 37:2893-2962). Paroxysmal AF, also known as intermittent or self-terminating AF, terminates within seven days of onset, either spontaneously or with intervention. Persistent AF is continuous AF that is sustained for more than seven days; pharmacologic or electrical cardioversion may be required to restore sinus rhythm. Long-standing persistent AF is continuous AF that is sustained for more than 12 months, and may not respond to medication or cardioversion. Permanent (chronic) AF is persistent AF where the patient and the doctor jointly decide to stop further attempts to restore and/or maintain sinus rhythm.

AF impacts left atrial function and geometry, and vice-versa. Over time, AF can result in decreased left atrial (LA) function (e.g., LA emptying fraction (LAEF)), as well as atrial remodeling (e.g., fibrosis and/or an increase in LA volumes that may become irreversible). Moreover, impaired LA function (e.g., LAEF) is associated with new-onset atrial fibrillation (Hirose et al., Eur Heart J. (2012) 13(3):243-50) as well as recurrence of AF after corrective procedures such as ablation. LA enlargement is strongly correlated with AF recurrence after electrical cardioversion. Impaired LA functional index (LAFI), calculated from LAEF, indexed maximal LA volume, and left ventricular outflow tract velocity time integral, is associated with adverse atrial remodeling, and increases the risk of developing incident AF and/or cardiovascular disease even when left atrial size is normal (Sardana et al., J Am Soc Echocardiogr. (2017) 30(9):904-12). LA parameters have been shown in observational studies to be powerful independent predictors of cardiovascular outcomes, including AF (Von Jeinsen et al., J Am Soc Echocardiograph. (2019) 33(1):72-81; Schaaf et al., Eur Heart J Cardiovasc Imaging (2017) 18:46-53).

AF often has comorbidity with heart failure. AF occurs in more than half of heart failure patients, while heart failure occurs in more than one third of AF patients. Heart failure (HF) is a clinical syndrome in which a patient's heart is unable to provide an adequate supply of blood flow to the body to meet the body's metabolic needs. For some patients with HF, the heart has difficulty pumping enough blood to support other organs in the body. Other patients may have a hardening or stiffening of the heart muscle itself, which blocks or reduces blood flow to the heart. Those two conditions result in inadequate blood circulation to the body and congestion of the lungs. HF can affect the right or left side of the heart, or both sides at the same time. It can be either an acute (short-term) or chronic (ongoing) condition. HF can be referred to as congestive HF when fluid builds up in various parts of the body. Symptoms include, but are not limited to, excessive fatigue, sudden weight gain, a loss of appetite, persistent coughing, irregular pulse, chest discomfort, angina, heart palpitations, edema (e.g., swelling of the lungs, arms, legs, ankles, face, hands, or abdomen), shortness of breath (dyspnea), protruding neck veins, and decreased exercise tolerance or capacity. AF and HF can cause and exacerbate each other, resulting in a significantly worse prognosis and increased mortality in comorbid patients.

Current therapies for AF include rate and rhythm control strategies and corrective procedures such as surgery (e.g., ablation) and sinus rhythm-restoring cardioversion. However, impaired LA function and geometry contribute to AF recurrence after corrective treatment; no current therapies directly address both depressed atrial function and atrial enlargement. Further, patients with concomitant AF and HF suffer from a significantly worse prognosis. There have been no effective therapies to treat comorbid AF and HF. In many cases, treatments shown to be effective for AF alone, or for HF alone, have poor efficacy (e.g., beta-blockers) and/or poor safety and tolerability profiles (e.g., Class I antiarrhythmic drugs) in patients with combined HF and AF (Kotecha et al., Eur Heart J (2015) 36:3250-7).

Accordingly, there remains a high medical need for new safe, well-tolerated, effective therapies for improving the atrial function of patients with AF, particularly when paired with systolic dysfunction such as reduced left ventricular ejection fraction (e.g., HFrEF).

### SUMMARY OF THE INVENTION

The present disclosure provides a method of treating atrial dysfunction in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I)

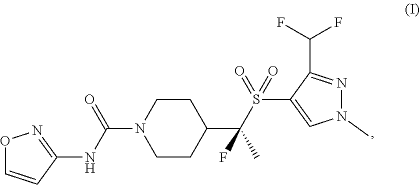 [SMILES: Cn1cc(S(=O)(=O)[C@@](C)(F)C2CCN(C(=O)Nc3ccon3)CC2)c(C(F)F)n1]

or a pharmaceutically acceptable salt thereof, optionally wherein the patient exhibits atrial fibrillation.

In one aspect, the present disclosure provides a method of treating atrial cardiomyopathy in a patient in need thereof (e.g., a patient who exhibits atrial dysfunction a patient who exhibits atrial fibrillation, etc.), comprising administering to the patient a therapeutically effective amount of Compound I, optionally wherein.

In one aspect, the present disclosure provides a method of treating atrial tachyarrhythmia in a patient in need thereof (e.g., a patient who exhibits atrial dysfunction, a patient who exhibits atrial fibrillation, etc.), comprising administering to the patient a therapeutically effective amount of Compound I.

In one aspect, the present disclosure provides a method of treating atrial fibrillation in a patient in need thereof, comprising administering to the patient a therapeutically effective amount of Compound I.

In one aspect, the present disclosure provides a method of reducing atrial fibrillation recurrence in a patient in need thereof, comprising administering to the patient a therapeutically effective amount of Compound I. In some embodiments, atrial fibrillation recurrence is reduced by 10% or greater (e.g., 20%, 30%, 40%, 50%, 60%, 70%, 80% or 90% or greater) in the patient.

In one aspect, the present disclosure provides a method of reducing atrial fibrillation burden in a patient in need thereof, comprising administering to the patient a therapeutically effective amount of Compound I. In some embodiments, atrial fibrillation burden is reduced by 10% or greater (e.g., 20%, 30%, 40%, 50%, 60%, 70%, 80% or 90% or greater) in the patient.

In one aspect, the present disclosure provides a method of reducing the duration of an atrial fibrillation episode in a patient in need thereof, comprising administering to the patient a therapeutically effective amount of Compound I. In some embodiments, the duration of the episode is reduced by 10% or greater (e.g., 20%, 30%, 40%, 50%, 60%, 70%, 80% or 90% or greater) in the patient.

In one aspect, the present disclosure provides a method of reducing the number of atrial fibrillation episodes during a monitoring period in a patient in need thereof, comprising administering to the patient a therapeutically effective amount of Compound I. In some embodiments, the number of atrial fibrillation episodes is reduced by 10% or greater (e.g., 20%, 30%, 40%, 50%, 60%, 70%, 80% or 90% or greater) in the patient.

In one aspect, the present disclosure provides a method of maintaining sinus rhythm in a patient in need thereof, comprising administering to the patient a therapeutically effective amount of Compound I. In some embodiments, the patient has sustained atrial tachyarrhythmia for 12 months or less (e.g., 9, 6, or 3 months or less) prior to the administering step. In some embodiments, the atrial tachyarrhythmia is atrial fibrillation.

In one aspect, the present disclosure provides a method of restoring sinus rhythm in a patient exhibiting atrial tachyarrhythmia, comprising administering to the patient a therapeutically effective amount of Compound I in combination with cardioversion (e.g., electrical cardioversion). In some embodiments, the atrial tachyarrhythmia is atrial fibrillation.

In one aspect, the present disclosure provides a method of preventing tachycardia-induced cardiomyopathy in a patient exhibiting atrial fibrillation, comprising administering to the patient a therapeutically effective amount of Compound I. In some embodiments, the tachycardia-induced cardiomyopathy is heart failure (e.g., heart failure with reduced ejection fraction (HFrEF)).

In some embodiments of the present methods, the patient has left atrial enlargement. In some embodiments, the present methods comprise selecting patients with left atrial enlargement for treatment with Compound I.

Also provided in the present disclosure are pharmaceutical compositions comprising Compound I and a pharmaceutically acceptable excipient; Compound I and the pharmaceutical compositions for use in any one of the treatment methods described herein; and the use of Compound I for the manufacture of a medicament for use in any of the treatment methods described herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a set of graphs showing the effect of Compound I on ATP turnover (ATPase) rates ex vivo in LV and LA swine myofibrils. Compound I increased ATP turnover (ATPase) rates in LV and LA swine myofibrils (Panel A), increasing Ca²⁺ sensitivity in fibers (Panels B and C; Panel B: LV tension/pCa curve) while preserving stiffness (Panel D). Panels A-D: mean±SEM. CTRL, control; LA, left atrial; LV, left ventricular; pCa, Ca²⁺ sensitivity.

FIG. 2 is a pair of graphs showing the effect of Compound I on SET and left atrial function and geometry in vivo in dogs with induced HF. Compound I prolonged SET, increasing indices of systolic LV function and stroke volume (Panel A), while decreasing size and improving performance in the LA (Panel B). Panels A and B: mean±SEM. 5HR, 5 hours post-treatment; LA, left atrial; LAEF, left atrial emptying fraction; LAFI, left atrial functional index; LV, left ventricular; LVFS, left ventricular fractional shortening; LVSV, left ventricular stroke volume; PRE, before dosing (i.e. baseline); SET, systolic ejection time.

FIG. 3 is a schematic diagram showing the design for an experiment on the effects of Compound 1 on AF inducibility and LA size and function in beagle dogs in the presence of phenylephrine. AFIB: atrial fibrillation. NSR: normal sinus rhythm. PE: phenylephrine.

FIG. 4 is a set of graphs showing the effect of Compound I on systolic blood pressure (SBP), left atrial minimal volume (LA Volₘᵢₙ), left atrial ejection fraction (LA EF), and duration of atrial fibrillation (AF_{duration}) in dogs that have undergone an AF inducibility protocol as described in Example 3. CPD I: Compound I. PACE: pace burst. PE: phenylephrine. PRE: pre-treatment.

FIG. 5 is a schematic diagram showing the design for an experiment on the effects of dobutamine on AF inducibility and LA size and function in beagle dogs in the presence of phenylephrine. AFIB: atrial fibrillation. NSR: normal sinus rhythm. PE: phenylephrine.

FIG. 6 is a set of graphs showing, from left to right, a comparison of left ventricular ejection fraction change (ΔEF) between Compound I and dobutamine, and the effect of dobutamine on left atrial minimal volume (LA Volₘᵢₙ), left atrial emptying fraction (LA EF), and duration of atrial fibrillation (AF_{duration}), in dogs that have undergone an AF inducibility protocol as described in Example 3. CPD I: Compound I. DOB: dobutamine. PE: phenylephrine. PRE: pre-treatment.

FIGS. 7A and 7B are schematic diagrams showing the clinical trial design for treating HFrEF with Compound I. BID, twice daily; MAD, multiple-ascending doses; SAD, single-ascending doses; SRC, Safety Review Committee.

FIG. 8 is a graph showing the LAFI change from baseline by Compound I plasma concentration. The line shown is from a non-parametric LOESS (locally estimated scatterplot smoothing) method.

### DETAILED DESCRIPTION OF THE INVENTION

The present disclosure provides methods, uses, and compositions relating to treating patients with atrial dysfunction (e.g., AF), including patients with comorbid atrial dysfunction and systolic dysfunction (impairment of the systolic function of the heart; e.g., reduced left ventricular ejection fraction such as HFrEF).

### Pharmaceutical Compositions

The pharmaceutical compositions used in the present therapies contain Compound I as an active pharmaceutical ingredient (API). Compound I refers to the compound (R)-4-(1-((3-(difluoromethyl)-1-methyl-1H-pyrazol-4-yl)sulfonyl)-1-fluoroethyl)-N-(isoxazol yl))piperidine-1-carboxamide, which has the following chemical structural formula (I):

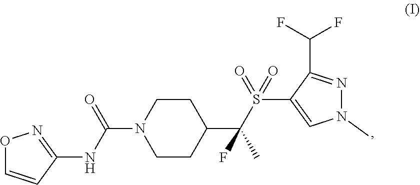 [SMILES: Cn1cc(S(=O)(=O)[C@@](C)(F)C2CCN(C(=O)Nc3ccon3)CC2)c(C(F)F)n1]

or a pharmaceutically acceptable salt thereof. Compound I is a myosin modulator that increases crossbridge formation (measured as phosphate release) between cardiac actin and myosin. Crossbridge formation and detachment are critical steps in each cycle of cardiac contraction. Compound I reversibly binds to myosin, increasing the number of myosin/actin crossbridges available to participate in the strongly bound state of the chemomechanical cycle and thereby increasing contraction. However, Compound I does not inhibit crossbridge detachment (measured as ADP release) and therefore does not affect any other states of the contraction cycle, nor does it affect calcium homeostasis. Compound I improves atrial function in part by improving (e.g., increasing) contractility of atrial cardiomyocytes (i.e., atrial contractility) without adversely affecting other important attributes of cardiovascular function.

The pharmaceutical compositions used herein may be provided in an oral dosage form (e.g., a liquid, a suspension, an emulsion, a capsule, or a tablet). In some embodiments, Compound I particles are compressed into tablets each containing 5, 25, 50, 75, 100, 125, 150, 175, or 200 mg of Compound I. In some embodiments, Compound I particles may be suspended in a suitable liquid such as water, a suspending vehicle, and/or flavored syrup for oral administration.

The Compound I API solid in the tablets or oral suspensions may have a mean particle size of, for example, 1-100, 1-50, or 15-50 μm in diameter (e.g., 1-5, 5-10, 1-10, 10-20, or 15-25 μm in diameter). In some embodiments, the Compound I has a mean particle size of no greater than 30, 25, 20, 15, 10, or 5 μm in diameter. In some embodiments, the Compound I API solid has a mean particle size of 15-25 μm in diameter for a particle size distribution (PSD) of D50 (i.e., 50% of the particles have a particle size of 15-25 μm in diameter). In certain embodiments, the Compound I has a mean particle size of 10 μm or less in diameter, e.g., D50 not more than (NMT) 10 μm. In certain embodiments, the Compound I has a mean particle size of 5 μm or less in diameter, e.g., D50 NMT 5 μm. The analysis of the particle size is typically carried out using a PSD method that is appropriate for determining the particle size of the primary particles. Ultrasound may be used to reduce agglomerates. The PSD technique used to measure particle size should not itself result in alteration of the primary particle size. In some of the Examples of the present disclosure, the PSD technique was performed with the Malvern Mastersizer 2000 with and without ultrasound.

Besides the Compound I API, the pharmaceutical compositions of the present disclosure may also contain pharmaceutically acceptable excipients. For example, the tablets used herein may contain bulking agents, diluents, binders, glidants, lubricants, and disintegrants. In some embodiments, Compound I tablets contain one or more of microcrystalline cellulose, lactose monohydrate, hypromellose, croscarmellose sodium, and magnesium stearate. The tablets may be coated to make them easier to ingest.

### Patient Populations

The therapies of the present disclosure may be used to treat a patient exhibiting atrial dysfunction. For example, the patient may exhibit atrial fibrillation. Abnormal atrial contractility, volume, function, and/or atrial cardiomyopathy may contribute to the atrial dysfunction.

The patient herein may be, for example, 18 years of age or older.

Left ventricular dysfunction is found in 20-30% of patients with AF. In some instances, the patient exhibits both atrial dysfunction (e.g., atrial fibrillation) and systolic dysfunction (also known as ventricular systolic dysfunction). The systolic dysfunction may be, for example, reduced left ventricular ejection fraction (e.g., HFrEF). The patient may or may not have received prior treatment for the atrial dysfunction and/or the systolic dysfunction. The volume of blood pumped by the heart is generally determined by: (a) the contraction of the heart muscle (i.e., how well the heart squeezes or its systolic function) and (b) the filling of the heart chambers (i.e., how well the heart relaxes and fills with blood or its diastolic function). Ejection fraction is used to assess the pump function of the heart; it represents the percentage of blood pumped from the left ventricle (the main pumping chamber) per beat. A normal or preserved ejection fraction is greater than or equal to 50 percent. If the systolic function of the heart is impaired such that the heart demonstrates substantial reduction in ejection fraction (i.e., an ejection fraction of <50%), this condition is known as heart failure with reduced ejection fraction (HFrEF). HFrEF with an ejection fraction of ≤40% is classical HFrEF, while HFrEF with an ejection fraction of 41-49% is classified as heart failure with mid-range ejection fraction (HFmrEF), under the 2013 American College of Cardiology Foundation/American Heart Association guidelines (Yancy et al., Circulation (2013) 128:e240-327) and the 2019 ACC Expert Consensus Decision Pathway on Risk Assessment, Management, and Clinical Trajectory of Patients Hospitalized With Heart Failure (Hollenberg et al., J Am Coll Cardiol (2019) 74:1966-2011). In certain embodiments, the patient exhibits both atrial dysfunction (e.g., atrial fibrillation) and diastolic dysfunction. In some cases, the patient exhibits atrial dysfunction (e.g., atrial fibrillation), systolic dysfunction, and diastolic dysfunction.

The atrial dysfunction being treated includes, without limitation, atrial cardiomyopathy (e.g., a left atrial myopathy) and atrial arrhythmia (e.g., atrial tachyarrhythmia) such as AF or atrial flutter. The atrial dysfunction (e.g., atrial tachyarrhythmia) may be acute or chronic. In certain embodiments, the patient may have sustained the atrial dysfunction (e.g., atrial tachyarrhythmia such as AF) continuously for a duration of, e.g., no more than 10 years, 9 years, 8 years, 7 years, 6 years, 5 years, 4 years, 3 years, 2 years, 12 months, 9 months, 6 months, 3 months, 1 month, 2 weeks, or 1 week prior to a therapy of the present disclosure.

In some embodiments, the patient has AF, which may be clinically manifested or may be subclinical (asymptomatic). Where AF cases are caused by a heart valve disorder, they are termed valvular AF. AF without a diagnosed heart valve disorder is called non-valvular AF. For example, in some embodiments non-valvular AF is AF in the absence of rheumatic mitral stenosis, a mechanical or bioprosthetic heart valve, or mitral valve repair. In terms of timing and duration, the AF being treated may be, e.g., paroxysmal, persistent, or long-standing persistent. In some cases, the AF is persistent but not long-standing persistent AF; that is, it has been sustained for 12 months or less. In certain embodiments, the patient has an AF burden of 1-70%, 2-70%, 3-70%, 1-99%, 2-99%, etc. Unless otherwise indicated, AF burden refers to the amount of AF that an individual has. In some embodiments, AF burden may be quantified as the percentage of time in which a patient is in AF during a monitoring period. In some embodiments, AF burden may be quantified as the duration of a patient's longest AF episode, or the number of AF episodes during a monitoring period.

In some embodiments, the patient additionally has one or more conditions selected from sleep apnea, hypertension, hyperlipidemia, hyperthyroidism, obesity, diabetes mellitus, glucose intolerance, alcohol use, tobacco use, prior myocardial infarction, chronic obstructive pulmonary disease, heart failure, coronary heart disease, rheumatic heart disease, valvular heart disease, nonvalvular heart disease, left ventricular hypertrophy, left ventricular diastolic dysfunction, and renal disease.

In some embodiments, the patient has a genetic predisposition to AF, such as an inherited cardiomyopathy or channelopathy.

In some embodiments, the patient has postoperative AF, i.e., new-onset AF in the period immediately following surgery (e.g., cardiac surgery).

In some embodiments, the patient has an implanted device with an atrial lead (e.g., pacemaker, ICD, CRT), or an implantable loop recorder (ILR).

In some embodiments, the patient has a Modified European Heart Rhythm Association (EHRA) symptom score of 1, 2a, 2b, 3, or 4, as defined in Table 1 below.

TABLE 1
Modified EHRA Symptom Scale

| Modified | | |
|---|---|---|
| EHRA | Patient | |
| Score | Symptoms | Description |
| 1 | None | AF does not cause any symptoms |
| 2a | Mild | Normal daily activity not affected by symptoms |
| | | related to AF |
| 2b | Moderate | Normal daily activity not affected by symptoms |
| | | related to AF, but patient troubled by symptoms |
| 3 | Severe | Normal daily activity affected by symptoms |
| | | related to AF |
| 4 | Disabling | Normal daily activity discontinued |

In some embodiments, the patient has been or is being treated with an anticoagulant, a rate control agent, or a rhythm control agent; or has undergone a physical intervention such as ablation (e.g., catheter ablation, surgical ablation, etc.) or cardioversion (e.g., electrical cardioversion or pharmacological cardioversion); or any combination thereof; but continues to exhibit AF symptoms. Such symptoms may include, e.g., heart palpitations, tachycardia, fatigue, dizziness, weakness, chest discomfort, reduced exercise capacity, increased urination, shortness of breath, angina, presyncope, syncope, sleeping difficulties, confusion, and psychosocial distress, or any AF symptom described herein.

In certain embodiments, the therapies of the present disclosure are used to treat a patient with atrial dysfunction (such as AF, e.g., paroxysmal or persistent AF), wherein the patient has any one or combination of the following:
an implanted device with an atrial lead (pace-maker, ICD, CRT), or an implantable loop recorder (ILR), wherein the device/ILR may have remote data transmission capability;
documented AF burden between 2 and 70% (e.g., over ≥2 continuous weeks);
clinical diagnosis of AF (based on electrocardiographic evidence), not due to transient conditions (e.g., post-operative, etc.); and
at least one episode of sustained AF within 6 months (based on medical records, or 12-lead ECG, or an episode of AF>10 minutes on Holter or patch, or prior electrical cardioversion) and without evidence of long-standing persistent or permanent AF.
   In some embodiments, the patient does not have any one or combination of the following:
   a) AF burden at screening <2% or >70%;
   b) AF with a reversible etiology (e.g., thyroid disease, alcohol, pulmonary embolism, early postoperative, acute pericarditis, trauma, etc.);
   c) pulmonary hypertension treated with pulmonary vasodilators (e.g., endothelin receptor antagonists, PDES inhibitors, etc.);
   d) known channelopathy, (e.g., long QT syndrome, Brugada syndrome, CPVT, etc.);
   e) long-standing persistent or permanent atrial fibrillation;
   f) atrial fibrillation diagnosed more than 10 years prior to the start of treatment;
   g) LA diameter >60 mm;
   h) catheter ablation within <6 months prior to the start of treatment, or planned or likely catheter ablation during treatment;
   i) introduction of new antiarrhythmic therapy <1 month prior to the start of treatment, or planned introduction of new antiarrhythmic therapy during treatment;
   j) electrical cardioversion performed <1 month prior to the start of treatment;
   k) heart failure of NYHA Class IV;
   l) symptomatic hypotension, or systolic blood pressure <90 mmHg, or diastolic blood pressure >95 mmHg;
   m) severe aortic valvular disease or mitral stenosis, planned or anticipated mitral clip or mitral valve repair during treatment, hypertrophic or infiltrative cardiomyopathy, active myocarditis, constrictive pericarditis, or clinically significant congenital heart disease;
   n) significant cardiovascular event within ≤90 days prior to the start of treatment, wherein the cardiovascular event is optionally acute coronary syndrome or stroke;
   o) cardiovascular intervention within ≤90 days prior to the start of treatment, wherein the cardiovascular intervention is optionally CABG, PCI, or valvular repair;
   p) device implantation within ≤45 days prior to the start of treatment, wherein the device is optionally a pacemaker or CRT;
   q) hospitalization for heart failure or treatment with IV inotropes within ≤90 days prior to the start of treatment;
   r) end stage heart failure; or
   s) life expectancy <6 months.

Where the patient exhibits systolic dysfunction in addition to a type of atrial dysfunction described herein (e.g., AF), the systolic dysfunction may be ventricular dysfunction, e.g., left ventricular dysfunction. The systolic dysfunction may be, for example, a syndrome or disorder selected from the group consisting of reduced left ventricular ejection fraction (LVEF), heart failure (e.g., heart failure with reduced ejection fraction (HFrEF), heart failure with preserved ejection fraction (HFpEF), congestive heart failure, or diastolic heart failure (with diminished systolic reserve)), cardiomyopathy (e.g., ischemic cardiomyopathy, dilated cardiomyopathy, hypertrophic cardiomyopathy (e.g., advanced hypertrophic cardiomyopathy), post-infarction cardiomyopathy, viral cardiomyopathy, toxic cardiomyopathy (optionally post-anthracycline anticancer therapy), metabolic cardiomyopathy (optionally cardiomyopathy in conjunction with enzyme replacement therapy), infiltrative cardiomyopathy (optionally amyloidosis), and diabetic cardiomyopathy), cardiogenic shock, a condition that benefits from inotropic support after cardiac surgery (e.g., ventricular dysfunction due to on-bypass cardiovascular surgery), myocarditis (e.g., viral myocarditis), atherosclerosis, secondary aldosteronism, myocardial infarction, valve disease (e.g., mitral regurgitation or aortic stenosis), systemic hypertension, pulmonary hypertension or pulmonary arterial hypertension, detrimental vascular remodeling, pulmonary edema, and respiratory failure.

The patient may experience systolic heart failure of the left ventricle, the right ventricle, or both ventricles. In some embodiments, the patient has right ventricular heart failure. In some embodiments, the patient has pulmonary hypertension (i.e., pulmonary arterial hypertension).

Systolic heart failure may be characterized by reduced ejection fraction, such as reduced left ventricular ejection fraction (e.g., less than about 50%, 45%, 40%, or 35%, including LVEF of 15-35%, 15-40% (e.g., 15-39%), 15-49%, 20-40%, 20-45%, 20-49%, 40-49%, and 41-49%) and/or increased ventricular end-diastolic pressure and volume.

In some embodiments, the patient has HFrEF (i.e., an ejection fraction of <50%). Heart failure with an ejection fraction of ≤40% is classical HFrEF, while heart failure with an ejection fraction of 41-49% is classified as heart failure with mid-range ejection fraction (HFmrEF). The patient may have a reduced left ventricular ejection fraction (LVEF) of less than 50%, e.g., less than 45%, 40%, 35%, 30%, 25%, 20%, or 15%. In certain embodiments, the patient has LVEF≤45% (e.g., 20-45%), ≤40% (e.g., 15-40%, 25-40%, 15-39%, or 25-39%), or ≤35% (e.g., 15-35%). The HFrEF may be of ischemic or non-ischemic origin, and may be chronic or acute.

In some embodiments, the patient has stable HF, e.g., stable HFrEF. As used herein, a patient who is “stable” with regard to a disease refers to a patient who has the disease and is not experiencing worsening of symptoms that might lead to a hospitalization or an urgent visit. For example, patients with stable HF can have impaired systolic function, but the symptoms of the dysfunction can be controlled or stabilized using available therapies.

In some embodiments, the patient has stable HFrEF (e.g., stable, chronic HFrEF of moderate severity), as defined by one or both of the following: (i) LVEF of less than 50%; and (ii) chronic medication for treatment of heart failure consistent with current guidelines, which may include at least one of a beta-blocker, an ACE inhibitor, an ARB, and an ARNI.

In some embodiments, the patient has paroxysmal or persistent AF with a normal left ventricular ejection fraction (e.g., greater than or equal to 50% and less than 60%). In certain embodiments, the patient has AF (e.g., paroxysmal or persistent) and heart failure with preserved ejection fraction (e.g., greater than or equal to 50% and less than 60%). In certain embodiments, the patient has AF (e.g., paroxysmal or persistent) and a normal left ventricular ejection fraction without heart failure.

In some embodiments, the therapies of the present disclosure may be used to treat a patient exhibiting dilated cardiomyopathy (DCM) (e.g., idiopathic DCM or genetic DCM). In certain embodiments, the patient has a dilated left or right ventricle, an ejection fraction less than 50% (e.g., ≤40%), and no known coronary disease. The DCM may be genetic DCM, wherein the patient has at least one genetic mutation in a sarcomeric contractile or structural protein that is known to cause DCM (see, e.g., Hershberger et al., Nat Rev Cardiol. (2013) 10(9):531-47 and Rosenbaum et al., Nat Rev Cardiol. (2020) 17(5):286-97), such as myosin heavy chain, titin, or troponin T. In some embodiments, the genetic mutation is in a gene selected from ABCC9, ACTC1, ACTN2, ANKRD1, BAG3, CRYAB, CSRP3, DES, DMD, DSG2, EYA4, GATAD1, LAMA4, LDB3, LMNA, MYBPC3, MYH6, MYH7, MYPN, PLN, PSEN1, PSEN2, RBM20, SCN5A, SGCD, TAZ, TCAP, TMPO, TNNC1, TNNI3, TNNT2, TPM1, TTN, VCL, or any combination thereof. For example, the genetic mutation is in a gene selected from ACTC1, DES, MYH6, MYH7, TNNC1, TNNI3, TNNT2, TTN, or any combination thereof. In particular embodiments, the genetic mutation is in the MYH7 gene or the TTN gene.

In some embodiments, the patient treated with a therapy described herein has been or is being treated with Entresto® and/or omecamtiv but continues to exhibit systolic heart failure symptoms. In some embodiments, the patient has been or is being treated with an ACE inhibitor or an ARB or an ARNI in conjunction with a beta blocker and optionally an aldosterone antagonist (wherein these agents may be, e.g., selected from those described herein), but continues to exhibit systolic heart failure symptoms.

In some embodiments, the patient treated with a therapy described herein has New York Heart Association (NYHA) Class I, II, III, or IV heart failure, as defined in Table 2 below. In certain embodiments, the patient has NYHA Class II-IV heart failure.

TABLE 2
New York Heart Association (NYHA) Classes of Heart Failure

| Class | Patient Symptoms |
|---|---|
| I | No limitation of physical activity. Ordinary physical activity does |
| | not cause undue fatigue, palpitation, dyspnea (shortness of breath). |
| II | Slight limitation of physical activity. Comfortable at rest. Ordinary |
| | physical activity results in fatigue, palpitation, dyspnea (shortness |
| | of breath). |
| III | Marked limitation of physical activity. Comfortable at rest. Less |
| | than ordinary activity causes fatigue, palpitation, or dyspnea. |
| IV | Unable to cany on any physical activity without discomfort. |
| | Symptoms of heart failure at rest. If any physical activity is |
| | undertaken, discomfort increases. |

The therapies of the present disclosure may be used to treat a patient with AF with or without systolic dysfunction (e.g., reduced left ventricular ejection fraction). In certain embodiments, the therapies of the present disclosure may be used to treat a patient with AF and reduced left ventricular ejection fraction of <50% (e.g., HFrEF). For example, the therapies may be used to maintain sinus rhythm (e.g., normal sinus rhythm) in a patient with AF and reduced left ventricular ejection fraction of <50% (e.g., HFrEF), and/or may be used to reduce atrial fibrillation recurrence in a patient with AF and reduced left ventricular ejection fraction of <50% (e.g., HFrEF). In particular embodiments, the patient has paroxysmal or persistent AF. In some instances, the therapies may be used to maintain sinus rhythm (e.g., normal sinus rhythm) in a patient with AF (e.g., paroxysmal or persistent AF), and/or may be used to reduce atrial fibrillation recurrence in a patient with AF (e.g., paroxysmal or persistent AF).

In some embodiments, the therapies of the present disclosure may be used to treat a patient with atrial dysfunction (e.g., AF), optionally in combination with reduced left ventricular ejection fraction (e.g., HFrEF), who exhibits mitral regurgitation. In some embodiments, the mitral regurgitation is chronic. In some embodiments, the mitral regurgitation is acute.

In certain embodiments, the therapies of the present disclosure are used to treat a patient with atrial dysfunction (such as AF, e.g., paroxysmal or persistent AF) and systolic dysfunction (e.g., reduced left ventricular ejection fraction such as HFrEF), wherein the patient has any one or combination of the following:

Documented reduced LVEF<50% within the past 12 months, and at least 30 days after

1) hospitalization for an event likely to decrease EF (e.g., acute coronary syndrome/myocardial infarction, sepsis, etc.);

2) an intervention likely to increase EF (e.g., cardiac resynchronization therapy, coronary revascularization); or

3) the first ever presentation for HF;

HFrEF with LVEF≤40%, wherein the patient is treated with any one or combination of a beta-blocker, angiotensin converting enzyme (ACE) inhibitor, angiotensin receptor blocker (ARB), and angiotensin receptor neprilysin inhibitor (ARNI);

NT-proBNP≥150 pg/mL at the start of therapy, or ≥100 pg/mL if the patient has a high BMI or is Black;

an implanted device with an atrial lead (pace-maker, ICD, CRT), or an implantable loop recorder (ILR), wherein the device/ILR may have remote data transmission capability;

documented AF burden between 2 and 70% (e.g., over ≥2 continuous weeks); and

clinical diagnosis of AF (based on electrocardiographic evidence), not due to transient conditions (e.g., post-operative, etc.), and

at least one episode of sustained AF within 6 months (based on medical records, or 12-lead ECG, or an episode of AF>10 minutes on Holter or patch, or prior electrical cardioversion) and without evidence of long-standing persistent or permanent AF.

In some embodiments, the patient does not have any one or combination of the following:
a) AF burden of <2% or >70%;
b) AF with a reversible etiology (e.g., thyroid disease, alcohol, pulmonary embolism, early postoperative, acute pericarditis, trauma, etc.);
c) pulmonary hypertension treated with pulmonary vasodilators (e.g., endothelin receptor antagonists, PDES inhibitors, etc.);
d) known channelopathy, (e.g., long QT syndrome, Brugada syndrome, CPVT, etc.);
e) long-standing persistent or permanent atrial fibrillation;
f) AF diagnosed more than 10 years prior to the start of treatment;
g) LA diameter >60 mm;
h) catheter ablation within <6 months prior to the start of treatment, or planned or likely catheter ablation during treatment;
i) introduction of new antiarrhythmic therapy <1 month prior to the start of treatment, or planned introduction of new antiarrhythmic therapy during treatment;
j) electrical cardioversion performed <1 month prior to the start of treatment;
k) heart failure of NYHA Class IV;
l) symptomatic hypotension, or systolic blood pressure <90 mmHg, or diastolic blood pressure >95 mmHg;
m) severe aortic valvular disease or mitral stenosis, planned or anticipated mitral clip or mitral valve repair during treatment, hypertrophic or infiltrative cardiomyopathy, active myocarditis, constrictive pericarditis, or clinically significant congenital heart disease;
n) significant cardiovascular event within ≤90 days prior to the start of treatment, wherein the cardiovascular event is optionally acute coronary syndrome or stroke;
o) cardiovascular intervention within ≤90 days prior to the start of treatment, wherein the cardiovascular intervention is optionally CABG, PCI, or valvular repair;
p) device implantation within ≤45 days prior to the start of treatment, wherein the device is optionally a pacemaker or CRT;
q) hospitalization for heart failure or treatment with IV inotropes within ≤90 days prior to the start of treatment;
r) end stage heart failure; or
s) life expectancy <6 months.

In some embodiments, a patient treated by a therapy described herein (e.g., a patient with atrial dysfunction and/or systolic dysfunction as described herein) has left atrial enlargement (LAE). In certain embodiments, a left atrium is considered enlarged if:
the left atrial diameter (LAD) is >4.1 cm in a male patient or >3.9 cm in a female patient;
the LAₘᵢₙVi is >19 mL/m²;
the LAₘₐₓVi is >41 mL/m²;
the LAEF is ≤45%; or
any combination of the above.

For example, the patient may have a LAD of 4.1-6.0 cm (male) or 3.9-6.0 cm (female). In some embodiments, the patient may have a LAD of 4.1-5.5 cm (male) or 3.9-5.5 cm (female). In certain embodiments, a patient may have a relatively mild left atrial enlargement (e.g., 4.1-4.6 cm (male) or 3.9-4.2 (female)). In certain embodiments, a patient may have a relatively moderate left atrial enlargement (e.g., 4.7-5.1 cm (male) or 4.3-4.6 cm (female)). In certain embodiments a patient may have a relatively severe left atrial enlargement (e.g., >5.2 cm (male) or ≥4.7 cm (female)). In some embodiments, the present treatment methods comprise the step of selecting patients with LAE for treatment with Compound I; the selection may be based on, for example, echocardiography.

A therapy described herein may include the step of selecting a patient with a type of atrial dysfunction as described herein (e.g., AF). In some embodiments, the patient is further selected as having a type of systolic dysfunction as described herein (e.g., reduced left ventricular ejection fraction such as HFrEF).

In some embodiments, a patient treated by a therapy described herein has previously been or is being treated for the atrial dysfunction and/or systolic dysfunction, with, for example, the standard of care for said condition(s), and has not shown adequate improvement with said treatment.

In some embodiments, a patient treated by a therapy described herein has previously been treated for AF with a therapeutic agent or intervention described herein. In certain embodiments, the patient has undergone ablation (e.g., catheter ablation) or cardioversion (e.g., electrical cardioversion), and accordingly is post-ablation or post-cardioversion.

### Treatment Regimens

The Compound I therapies described herein may treat atrial dysfunction (e.g., AF) in a patient. In certain embodiments, the patient may also have systolic dysfunction such as reduced left ventricular ejection fraction (e.g., HFrEF). The patient may receive a therapy of the present disclosure for at least one month, at least six months, at least twelve months, at least one year, or longer, or until such time the patient no longer needs the treatment.

In some embodiments of the present therapies, Compound I is administered in a total daily oral amount of 10-700 mg (e.g., 50-150 mg). For example, Compound I may be administered in a total daily oral amount of 10, 25, 50, 75, 100, 125, 150, 175, 200, 250, 300, 350, 400, 450, 500, 525, 550, 600, or 700 mg. As another example, Compound I may be administered in a total daily oral amount of 50, 100, or 150 mg. In one embodiment, Compound I is orally administered at 10-175 mg BID (twice daily) (e.g., 10, 25, 30, 35, 37.5, 40, 45, 50, 55, 60, 62.5, 65, 70, 75, 80, 85, 90, 95, 100, 105, 110, 115, 120, 125, 130, 135, 140, 145, 150, 155, 160, 165, 170 or 175 mg). For example, Compound I may be orally administered at 10-75 mg (e.g., 10 mg, 25 mg, 50 mg, or 75 mg) BID. In another embodiment, Compound I is orally administered at 25-350 mg QD (once daily) (e.g., 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, or 350 mg). For example, Compound I may be orally administered at 50-150 mg (e.g., 50 mg, 100 mg, or 150 mg) QD. The intervals between BID doses are, for example, between approximately 10-12 hours apart when possible (e.g., morning and evening).

As used herein, administration of Compound I or a pharmaceutical composition containing Compound I (“Compound I medication”) includes self-administration by the patient himself or herself (e.g., oral intake by the patient).

In some embodiments, a patient orally consumes a loading dose of Compound I with or without food followed by a maintenance dose (e.g., a dose described above) approximately 10-12 hours thereafter with or without food, and then continues his/her daily recommended maintenance dose regimen with or without food (e.g., morning and evening for BID dosing regimens). The loading dose, may be for example, 1.5-fold the maintenance dose for a QD dosing regimen or 2-fold for a BID dosing regimen. In some embodiments, the loading dose is 50-250 mg of Compound I, e.g., for a maintenance dosing of 25-75 mg BID or 50-150 mg QD.

In some embodiments, Compound I absorption by the patient may be facilitated by food. In some embodiments, the food is high in fat content; that is, more than 50% of the calories of the food are derived from fat). In some embodiments, where Compound I is taken with food (e.g., high fat food), the mean particle size of the Compound I API is over 15 μm in diameter and the QD dose is greater than approximately 200 mg. In some embodiments, the total daily dose of Compound I needed by a patient if the medication is taken in a fed state (e.g., within about two hours of food, within about one and a half hours of food, or within about one hour of food) may be lower than the total daily dose needed by the patient if the medication is taken not in a fed state. “Within about X hours of food” means about X hours before the start or after the end of ingestion of food.

In certain embodiments, Compound I tablets or capsules are taken orally by the patient—with food or within about two hours of food (e.g., within about one and a half hours of food or within about one hour of food). In some embodiments, the patient takes the medication orally once daily with meals. In some embodiments, the patient takes the medication twice daily with meals. For example, the patient may take the medication at breakfast and dinner. In some embodiments, the medication may be taken with a glass of drink such as water or milk (e.g., whole milk) if desired.

In some embodiments, the Compound I API in the medication is micronized and has a mean particle size of 10 μm or less in diameter (D50 not more than (NMT) 10 μm), or of 5 μm or less in diameter (D50 NMT 5 μm). In certain embodiments, when Compound I particles in the medication have D50 NMT 5 or 10 μm, the medication may be taken orally by a patient twice a day (e.g., every 10-12 hours, or morning and evening), with or without food.

The dosage used for a particular patient may be adjusted based on the patient's condition and/or the patient's unique PK profile. Current studies indicate that the drug dosages and exposures tested are safe and are well tolerated. In some embodiments, Compound I may be administered to the patient at a dose that results in plasma concentrations of 1000 to 8000 ng/mL (e.g., 1000-2000 ng/mL, 1500-3000 ng/mL, 2000-3000 ng/mL, 3000-4000 ng/mL, 3000-4500 ng/mL, 3500-5000 ng/mL, 4000-5000 ng/mL, 5000-6000 ng/mL, 6000-7000 ng/mL, or 7000-8000 ng/mL). In some embodiments, Compound I may be administered to the patient at a dose that results in plasma concentrations of <2000, 2000-3500, or ≥3500 ng/mL (e.g., 2000-3500 ng/mL). In some embodiments, Compound I may be administered to the patient in amounts that result in a plasma Compound I concentration of greater than 1500, 2000, 2250, 2500, 2750, 3000, 3500, 4000, 5000, 6000, or 7000 ng/mL. In some embodiments, the Compound I target plasma concentration is between 1000-4000 ng/mL. In certain embodiments, the Compound I target plasma concentration is between 1500-3500 ng/mL. In particular embodiments, the Compound I target plasma concentration is between 2000-3500 ng/mL. The Compound I plasma concentration may be determined by any method known in the art, such as, for example, high performance liquid chromatography (HPLC), liquid chromatography-mass spectroscopy (LC-MS such as high performance LC-MS), gas chromatography (GC), or any combination thereof.

In some embodiments, the therapies described herein comprise monitoring the patient for an adverse event such as headache, lethargy, chest discomfort, bradycardia, heart block, sinus tachycardia, ventricular tachycardia, palpitation, cardiac arrhythmia, increase in NT-proBNP levels, increase in troponin levels, and cardiac ischemia. If a severe adverse event occurs, the patient may be treated for the adverse event, and/or may discontinue treatment with Compound I.

### Combination Therapy

The present disclosure provides both Compound I monotherapy and combination therapy. In combination therapy, a Compound I regimen of the present disclosure is used in combination with an additional therapy regimen, e.g., a guideline-directed medical therapy (GDMT), also referred to as a standard of care (SOC) therapy, for one or more cardiac conditions exhibited by the patient, or other therapy useful for treating the relevant disease or disorder. The additional therapeutic agent may be administered by a route and in an amount commonly used for said agent or at a reduced amount, and may be administered simultaneously, sequentially, or concurrently with Compound I.

In some embodiments, Compound I is administered on top of the SOC for a condition of atrial dysfunction, such as atrial fibrillation; a condition of systolic dysfunction, such as systolic heart failure and/or reduced left ventricular ejection fraction; or both.

In certain embodiments, the patient exhibiting atrial dysfunction (e.g., atrial fibrillation) is given, in addition to the Compound I medication, another therapeutic agent for treating the atrial dysfunction. In some embodiments, the therapeutic agent is an antithrombotic agent (e.g., an anticoagulant such as a NOAC), a rate control agent, an antiarrhythmic agent (e.g., a Class Ia, Ic, or III antiarrhythmic agent), a pharmacological cardioversion agent, a RAAS inhibitor, etc. In some embodiments, the Compound I medication is administered to a patient who has had or plans to have a non-pharmacological intervention such as electrical cardioversion, left atrial appendage occlusion (e.g., using a Watchman device) or excision, atrioventricular nodal ablation (e.g., with permanent ventricular pacing), catheter ablation, surgical ablation (e.g., Maze procedure), hybrid catheter and surgical ablation, pulmonary vein ablation, or a permanent pacemaker. Any combination of the above agents and interventions is also contemplated.

In some embodiments, the Compound I medication is administered to the patient in place of an antiarrhythmic agent. The patient may have had prior treatment with an antiarrhythmic agent that is then replaced by the Compound I medication, or the patient may be treated with the Compound I medication without prior treatment with an antiarrhythmic agent.

In some embodiments, a patient with atrial dysfunction (e.g., AF) is treated with ablation (e.g., catheter ablation, surgical ablation, etc.) in addition to the Compound I medication. In certain cases, the patient is treated with the Compound I medication post-ablation (e.g., post-catheter ablation).

In some embodiments, a patient with atrial dysfunction (e.g., AF) is treated with an anticoagulant (e.g., a NOAC) in combination with a rate control agent (e.g., a beta-blocker, digoxin, and/or amiodarone) in addition to the Compound I medication.

In some embodiments, a patient with atrial dysfunction (e.g., AF) is treated with cardioversion (e.g., electrical cardioversion) in addition to the Compound I medication. In certain cases, the patient is treated with the Compound I medication post-cardioversion (e.g., post-electrical cardioversion).

In some embodiments, a patient with atrial dysfunction (e.g., AF) is treated with cardioversion (e.g., electrical cardioversion) in combination with an antiarrhythmic drug (e.g., amiodarone, sotalol, or dofetilide) in addition to the Compound I medication.

In some embodiments, a patient with atrial dysfunction (e.g., AF) is treated with ablation (e.g., catheter ablation, surgical ablation, etc.) and antiarrhythmic medication.

In certain embodiments, the patient exhibiting systolic dysfunction (e.g., reduced left ventricular ejection fraction such as HFrEF) in addition to the atrial dysfunction (e.g., AF) is given, in addition to the Compound I medication and optionally a therapeutic agent for treating the atrial dysfunction as described herein, another therapeutic agent for treating the systolic dysfunction. In some embodiments, the therapeutic agent is a beta-blocker, an angiotensin converting enzyme (ACE) inhibitor, an angiotensin receptor antagonist (e.g., an angiotensin II receptor blocker), an angiotensin receptor neprilysin inhibitor (ARNI) (e.g., sacubitril/valsartan), a mineralocorticoid receptor antagonist (e.g., an aldosterone antagonist), a cholesterol lowering drug (e.g., a statin), an If channel inhibitor (e.g., ivabradine), a neutral endopeptidase inhibitor (NEPi), a positive inotropic agent, potassium or magnesium, a proprotein convertase subtilisin kexin-type 9 (PCSK9) inhibitor, a vasodilator, a diuretic (e.g., a loop diuretic such as furosemide), a RAAS inhibitor, a soluble guanylate cyclase (sGC) activator or modulator (e.g., vericiguat), an SGLT2 inhibitor (e.g., dapagliflozin), an antiarrhythmic medication, an anticoagulant, an antithrombotic agent, an antiplatelet agent, or any combination thereof. In particular embodiments, the patient is treated with an ARNI, a beta blocker, and/or an MRA in addition to the Compound I medication. In certain embodiments, the ARNI, beta blocker, and/or MRA are selected from those described herein, in any combination. In particular embodiments, the patient is treated with an ACE inhibitor and/or ARB and/or ARNI, in conjunction with a beta blocker and optionally an aldosterone antagonist, in addition to the Compound I medication. In certain embodiments, the ACE inhibitor, ARB, ARNI, beta blocker, and/or aldosterone antagonist are selected from those described herein, in any combination.

In some embodiments, a patient with atrial dysfunction (e.g., AF) and systolic dysfunction (e.g., reduced LVEF such as HFrEF) is treated with catheter ablation in addition to Compound I medication.

In some embodiments, a patient with atrial dysfunction (e.g., AF) and systolic dysfunction (e.g., reduced LVEF such as HFrEF) is treated with an anticoagulant (e.g., a NOAC) in combination with a rate control agent (e.g., a beta-blocker, digoxin, and/or amiodarone) in addition to the Compound I medication.

In some embodiments, a patient with atrial dysfunction (e.g., AF) and systolic dysfunction (e.g., reduced LVEF such as HFrEF) is treated with electrical cardioversion in combination with an antiarrhythmic drug (e.g., amiodarone, sotalol, or dofetilide) in addition to the Compound I medication.

In some embodiments, a patient with atrial dysfunction (e.g., AF) and systolic dysfunction (e.g., reduced LVEF such as HFrEF) is treated with cardioversion, an anticoagulant, a diuretic, a rate control agent, a RAAS antagonist, and a rhythm control agent in addition to the Compound I medication.

In some embodiments, a patient with atrial dysfunction (e.g., AF) and systolic dysfunction (e.g., reduced LVEF such as HFrEF) is treated with an anticoagulant; a diuretic; and an angiotensin-converting enzyme (ACE) inhibitor, angiotensin II receptor blocker (ARB), and/or mineralocorticoid receptor antagonist in addition to the Compound I medication.

In some embodiments, a patient with atrial dysfunction (e.g., AF) and systolic dysfunction (e.g., reduced LVEF such as HFrEF) is treated with an ARNI such as sacubitril/valsartan (Entresto®) or a sodium-glucose cotransporter 2 inhibitor (SGLT2i) such as empaglifozin (e.g., Jardiance®), dapagliflozin (e.g., Farxiga®), canagliflozin (e.g., Invokana®), or sotagliflozin, in addition to the Compound I medication.

In some embodiments, a patient with atrial dysfunction (e.g., AF) and systolic dysfunction (e.g., reduced LVEF such as HFrEF) is treated with an ARNI, a beta blocker, and/or an MRA in addition to the Compound I medication.

In some embodiments, a patient with atrial dysfunction (e.g., AF) and systolic dysfunction (e.g., reduced LVEF such as HFrEF) is treated with an ACE inhibitor and/or ARB and/or ARNI, in conjunction with a beta blocker and optionally an aldosterone antagonist in addition to the Compound I medication.

In some embodiments, a patient with systolic dysfunction (e.g., reduced LVEF such as HFrEF) is treated with an ACE inhibitor or ARB in combination with the Compound I medication to prevent new-onset AF.

In some embodiments, Compound I is administered to a patient with atrial dysfunction (e.g., AF) on top of the SOC for HFrEF in combination with AF; for example, SOC according to the CAN-TREAT algorithm (Kotecha et al., Eur Heart J. (2015) 36:3250-7). The algorithm involves Cardioversion, Anticoagulation (e.g., with vitamin K antagonists such as warfarin, or NOACs), Normalization of fluid balance (e.g., with diuretics), Targeting initial heart rate <110 bmp (e.g., with beta blockers or digoxin), Renin-angiotensin-aldosterone system modulation (e.g., with ACE inhibitors, ARB, and/or mineralocorticoid receptor antagonists), Early consideration of rhythm control (e.g., using antiarrhythmic agents such as amiodarone and/or dofetilide, cardioversion, and/or catheter ablation), Advanced heart failure therapies (e.g., resynchronization therapy), and Treatment of other CV diseases such as ischemia and hypertension.

Suitable angiotensin converting enzyme (ACE) inhibitors may include, e.g., captopril, enalapril, fosinopril, lisinopril, perindopril, quinapril, ramipril, and trandolapril.

Suitable antiarrhythmic medications (rhythm control agents) may include, e.g., amiodarone, dronedarone, propafenone, flecainide, dofetilide, ibutilide, quinidine, procainamide, disopyramide, and sotalol. In some embodiments, the antiarrhythmic medications are of Class Ia, Ic, or III.

Suitable anticoagulants may include, e.g., warfarin, apixaban, rivaroxaban, edoxaban, and dabigatran. In some embodiments, the anticoagulants are oral anticoagulants (OACs); in certain embodiments, OACs may be administered with vitamin K antagonists. In some embodiments, the anticoagulants are non-vitamin K oral anticoagulants (NOACs). In some embodiments, the anticoagulants are vitamin K antagonists (e.g., warfarin, acenocoumarol, phenprocoumon, etc.).

Suitable ARBs may include, e.g., A-81988, A-81282, BIBR-363, BIBS39, BIBS-222, BMS-180560, BMS-184698, candesartan, candesartan cilexetil, CGP-38560A, CGP-48369, CGP-49870, CGP-63170, CI-996, CV-11194, DA-2079, DE-3489, DMP-811, DuP-167, DuP-532, E-4177, elisartan, EMD-66397, EMD-73495, eprosartan, EXP-063, EXP-929, EXP-3174, EXP-6155, EXP-6803, EXP-7711, EXP-9270, FK-739, GA-0056, HN-65021, HR-720, ICI-D6888, ICI-D7155, ICI-D8731, irbesartan, isoteoline, KRI-1177, KT3-671, KW-3433, losartan, LR-B/057, L-158809, L-158978, L-159282, L-159874, L-161177, L-162154, L-163017, L-159689, L-162234, L-162441, L-163007, LR-B/081, LR B087, LY-285434, LY-302289, LY-315995, LY-235656, LY-301875, ME-3221, olmesartan, PD-150304, PD-123177, PD-123319, RG-13647, RWJ-38970, RWJ-46458, saralasin acetate, S-8307, S-8308, SC-52458, saprisartan, saralasin, sarmesin, SL-91.0102, tasosartan, telmisartan, UP-269-6, U-96849, U-97018, UP-275-22, WAY-126227, WK-1492.2K, YM-31472, WK-1360, X-6803, valsartan, XH-148, XR-510, YM-358, ZD-6888, ZD-7155, ZD-8731, and zolasartan.

Suitable mineralocorticoid receptor antagonists include, e.g., aldosterone inhibitors such as potassium-sparing diuretics. Examples include, e.g., eplerenone, spironolactone, and canrenone.

Suitable pharmacological cardioversion agents include, e.g., flecainide, dofetilide, propafenone, amiodarone, ibutilide, vernakalant, etc.

Suitable positive inotropic agents include, e.g., digoxin, pimobendan, beta adrenergic receptor agonists such as dobutamine, phosphodiesterase (PDE)-3 inhibitors such as milrinone, and calcium-sensitizing agents such as levosimendan.

Suitable rate control agents include, e.g., beta-blockers, non-dihydropyridine calcium channel blockers (e.g., verapamil, diltiazem), digoxin, digitoxin, digitalis, and amiodarone. Suitable beta-blockers include, e.g., bisoprolol, carvedilol, carvedilol CR, atenolol, esmolol, landiolol, nebivolol, propranolol, nadolol, metaprolol tartrate, and metoprolol succinate extended release (metoprolol CR/XL)).

Suitable vasodilators include, e.g., phosphodiesterase inhibitors, endothelin receptor antagonists, renin inhibitors, smooth muscle myosin modulators, isosorbide dinitrate, and hydralazine. In the case of atrial dysfunction, a calcium channel blocker may be used.

In some embodiments, Compound I is administered in combination with lifestyle changes such as reducing alcohol or caffeine intake, quitting smoking, limiting stimulants, achieving or maintaining a healthy weight, physical activity, treating sleep apnea, and/or controlling high blood pressure and/or blood sugar levels, or any combination thereof.

If any adverse effect occurs, the patient may be treated for the adverse effect. For example, a patient experiencing headache due to the Compound I treatment may be treated with an analgesic such as ibuprofen and acetaminophen.

### Treatment Outcomes

The therapies of the present disclosure treat and/or ameliorate atrial dysfunction. In some embodiments, the therapies also treat and/or ameliorate systolic dysfunction. As used herein, the terms “treat,” “treating” and “treatment” refer to any indicia of success in the treatment or amelioration of a pathology, injury, condition, or symptom related to the dysfunction, including any objective or subjective parameter such as abatement; remission; diminishing of symptoms; making the pathology, injury, condition, or symptom more tolerable to the patient; decreasing the frequency or duration of the pathology, injury, condition, or symptom; or, in some situations, delaying or preventing the onset of the pathology, injury, condition, or symptom. Treatment or amelioration can be based on any objective or subjective parameter, including, e.g., the result of a physical examination. For example, treatment of atrial dysfunction (e.g., AF) encompasses, but is not limited to, any one or combination of: improving atrial myocyte contractility, improving atrial contractility, improving atrial cardiomyopathy, improving atrial arrhythmia (e.g., tachyarrhythmia), reducing AF recurrence, reducing AF burden, preventing incident AF, maintaining sinus rhythm (e.g., after cardioversion), restoring sinus rhythm (e.g., in combination with cardioversion), decreasing left atrial volume (e.g., minimum or maximum volume), increasing left atrial emptying fraction, increasing left atrial functional index, and alleviating or preventing the symptoms of atrial dysfunction. Symptoms of atrial dysfunction (e.g., AF) may include, e.g., heart palpitations, tachycardia, fatigue, dizziness, weakness, chest discomfort, reduced exercise capacity, increased urination, shortness of breath, angina, presyncope, syncope, sleeping difficulties, confusion, and psychosocial distress. Treatment of systolic dysfunction encompasses, but is not limited to, any one or combination of improving the cardiac functions of the patient and alleviating or preventing the symptoms of systolic heart failure (especially during exercise, including walking or stair climbing). Symptoms of systolic heart failure may include, e.g., dyspnea (e.g., orthopnea, paroxysmal nocturnal dyspnea), coughing, cardiac asthma, wheezing, hypotension, dizziness, confusion, cool extremities at rest, pulmonary congestion, chronic venous congestion, ankle swelling, peripheral edema or anasarca, nocturia, ascites, hepatomegaly, jaundice, coagulopathy, fatigue, exercise intolerance, jugular venous distension, pulmonary rales, peripheral edema, pulmonary vascular redistribution, interstitial edema, pleural effusions, and fluid retention.

In some embodiments, the therapies of the present disclosure reduce AF burden and/or AF recurrence in a patient (e.g., a patient from a population described herein). AF burden and/or AF recurrence may be reduced by 10% or greater. In some embodiments, AF burden and/or AF recurrence are reduced by 5%, 10%, 15%, 20%, 25%, 30%, 35%, 40%, 45%, 50%, 55%, 60%, 65%, 70%, 75%, 80%, 85%, 90%, or 95% or greater, or 100%. In some embodiments, the percentage of time the patient spends in AF during a monitoring period is reduced by 5%, 10%, 15%, 20%, 25%, 30%, 35%, 40%, 45%, 50%, 55%, 60%, 65%, 70%, 75%, 80%, 85%, 90%, or 95% or greater, or 100%. In some embodiments, the therapies reduce the duration of a patient's longest AF episode, or the number of AF episodes during a monitoring period, e.g., by 5%, 10%, 15%, 20%, 25%, 30%, 35%, 40%, 45%, 50%, 55%, 60%, 65%, 70%, 75%, 80%, 85%, 90%, or 95% or greater, or 100%. In some embodiments, the monitoring period may be on the order of minutes (e.g., 10 minutes, 20 minutes, 30 minutes, 40 minutes, 50 minutes or more; 10 minutes to 59 minutes), hours (e.g., 1 hour, 2 hours, 4 hours, 6 hours, 8 hours, 12 hours, 18 hours or more; 1 hour to 24 hours) days (e.g., 1 day, 2 days, 3 days, 4 days, 5 days, or 6 days or more), weeks (e.g., 1 week, 2 weeks, 4 weeks, 8 weeks, 12 weeks, 16 weeks, 20 weeks, 24 weeks, 32 weeks, 40 weeks or more), or years. For instance, the monitoring period may be 24 hours, 1 week, 2 weeks, 1 month, 2 months, 3 months, 4 months, 5 months, 6 months, 1 year, or more.

In some embodiments, the therapies of the present disclosure maintain sinus rhythm (e.g., normal sinus rhythm) in a patient (e.g., a patient from a population described herein). In certain embodiments, the patient has been treated with or will be treated with cardioversion (e.g., electrical cardioversion). In some embodiments, the therapies of the present disclosure, in combination with cardioversion (e.g., electrical cardioversion), restore sinus rhythm (e.g., normal sinus rhythm) in a patient. In some embodiments, sinus rhythm is maintained for at least one, two, three, four, five, six, or seven days; at least one, two, three, or four weeks; at least one, two, three, four, five, six, nine, or twelve months; at least 1, 2, 3, 4, 5, 6, 7, 8, 9, 10 years; or longer; or until such time that the patient no longer needs the treatment.

In some embodiments, the therapies of the present disclosure reduce the risk of, or delay the incidence of, myocardial infarction, ventricular arrhythmia, heart failure, chronic kidney disease, end-stage renal disease, sudden cardiac death, or all-cause death in a patient.

In some embodiments, the therapies of the present disclosure improve the patient's quality of life, as measured by the 6-Month Walk Test (6-MWT), Kansas City Cardiomyopathy Questionnaire (KCCQ), Atrial Fibrillation Effect on Quality-of-Life (AFEQT) measure, and/or Mayo AF-Specific Symptom Inventory (MAFSI).

In some embodiments, the therapies of the present disclosure may prevent or delay tachycardia-induced cardiomyopathy in a patient exhibiting atrial fibrillation. In certain embodiments, the tachycardia-induced cardiomyopathy is heart failure (e.g., HFrEF).

In some embodiments, the therapies of the present disclosure may prevent or delay incident AF (initial occurrence of AF) in a patient. Additionally or alternatively, the therapies may prevent or delay AF recurrence in a patient. In certain embodiments, the patient has systolic dysfunction such as chronic heart failure (e.g., HFrEF for three months or more). In certain embodiments, the patient has left atrial enlargement. In some instances, the patient has systolic dysfunction and left atrial enlargement.

In some embodiments, the therapies of the present disclosure prevent or delay AF progression in a patient. For example, the therapies may prevent or delay a patient's progression from paroxysmal to persistent AF, or from paroxysmal or persistent AF to long-standing persistent or permanent AF. In certain embodiments, the patient has systolic dysfunction such as chronic heart failure (e.g., HFrEF for three months or more). In certain embodiments, the patient has left atrial enlargement. In some instances, the patient has systolic dysfunction and left atrial enlargement.

Pharmacodynamic (PD) parameters that can be used to measure the atrial functions of a patient are shown in Table 3 below. These PD parameters are routinely used by clinicians and can be measured by standard transthoracic echocardiogram.

TABLE 3
Transthoracic Echocardiography (TTE) Parameters

| Abbreviation | Parameter |
|---|---|
| LAEF | Left Atrial Emptying Fraction |
| LAₘₐₓVi | Left Atrial Maximum Volume Index |
| LAₘᵢₙVi | Left Atrial Minimum Volume Index |
| LAFI | Left Atrial Functional Index |

In some embodiments, the therapies of the present disclosure:
increase LAEF in the patient by 5%, 10%, 15%, 20%, 25%, 30%, 35%, 40%, 45%, 50%, or more;
decrease LAₘᵢₙVi in the patient by 5%, 10%, 15%, 20%, 25%, 30%, 35%, 40%, 45%, 50%, or more;
decrease LAₘₐₓVi in the patient by 5%, 10%, 15%, 20%, 25%, 30%, 35%, 40%, 45%, 50%, or more; and/or
increase LAFI in the patient by 5%, 10%, 15%, 20%, 25%, 30%, 35%, 40%, 45%, 50%, or more.
   In certain embodiments, the patient may have left atrial enlargement prior to therapy.

The present therapies may reduce the risk of cardiovascular death, and/or the risk, frequency, or duration of hospitalization/urgent care visits, for a patient population described herein. The hospitalization and urgent care visits may be for atrial dysfunction as described herein, systolic dysfunction as described herein, or both. In some embodiments, “reducing the risk” of an event means increasing the time to the event by at least 10% (e.g., at least 15%, 20%, 30%, 40%, 50%, 60%, 70%, 80%, 90%, 100%, or more). The risk can be relative risk or absolute risk. In some embodiments, the present therapies reduce the frequency of hospitalization and urgent care visits by at least 10% (e.g., at least 15%, 20%, 30%, 40%, 50%, 60%, 70%, 80%, 90%, or 100%). In some embodiments, the present therapies reduce the duration of hospitalization by at least 10% (e.g., at least 15%, 20%, 30%, 40%, 50%, 60%, 70%, 80%, 90%, or 100%).

The advantages of the present therapies include the features that the treatment

(i) has minimal impact on relaxation (e.g., no more than a modest increase in systolic ejection time and no discernable effect on diastolic function), calcium homeostasis, or troponin level (e.g., no more than a mild elevation of troponin);

(ii) does not impair ADP release;

(iii) does not change cardiac phase distribution;

(iv) has no more than a modest effect on SET;

(v) does not cause drug-related cardiac ischemia (e.g., as determined by clinical symptoms, ECG, cardiac biomarkers such as troponin, creatine kinase-muscle/brain (CK-MB), cardiac imaging, and coronary angiograms);

(vi) does not cause drug-related atrial or ventricular arrhythmia;

(vii) does not cause drug-induced liver injury as measured by alanine aminotransferase or aspartate aminotransferase, bilirubin; and

(viii) does not result in abnormalities in the patient's urine, serum, blood, systolic blood pressure, diastolic blood pressure, pulse, body temperature, blood oxygen saturation, or electrocardiography (ECG) readings.

### Articles of Manufacture and Kits

The present invention also provides articles of manufacture, e.g., kits, comprising one or more dosages of the Compound I medication, and instructions for patients (e.g., for treatment in accordance with a method described herein). The articles of manufacture may also contain an additional therapeutic agent in the case of combination therapy. Compound I tablets or capsules may be blistered and then carded, produced with, for example, 5-20 tablets per blister card; each tablet or capsule may contain 5, 25, 50, 75, or 100 mg of Compound I, and such blister card may or may not additionally include a loading dose tablet or capsule. The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Ex Vivo Biochemical Studies of the Effect of Compound I on Left Atrial Function

This example describes non-clinical studies of the effects of Compound I on left atrial function.

### Materials and Methods

### ATP Turnover Rate Studies

The ability of Compound I to increase myocardial ATP turnover rates selectively was evaluated using both left atrial (LA) and left ventricular (LV) myofibrils prepared from Yucatan mini-pig hearts, as well as subfragment-1 (51) myosins from cardiac (recombinant human), skeletal (rabbit psoas) and smooth (chicken gizzard) muscles. Pig hearts were harvested, and the left atria/ventricles were freshly excised, dissected, frozen in liquid nitrogen and stored at −80° C. Myofibrils and S1 myosins were prepared as described in Kawas et al., J Biol Chem. (2017) 292(40:16571-7. Both atrial (n=4 hearts, 2 replicates each) and ventricular (n=3 hearts, 2 replicates each) myofibrils were assayed at a constant concentration of 1.0 mg/mL (at Ca²⁺ sensitivity [pCa] 6.0). Rabbit skeletal (0.2 μM, n=8), chicken gizzard (0.5 n=10) and recombinant human cardiac (0.5 n=10) S1 myosin were assayed with a constant concentration of actin (14 μM).

Steady-state ATPase measurements at varying concentrations of Compound I (0-50 μM, in 2% DMSO) were conducted using a coupled enzyme system utilizing pyruvate kinase and lactate dehydrogenase. This enzyme system couples the formation of ADP to the oxidation of NADH leading to an absorbance change at 340 nm. The buffering system used in all experiments was 12 mM PIPES, 2 mM MgCl₂ and 1 mM dithiothreitol (DTT) at pH 6.8 (PM12 buffer). All measurements were carried out at 25° C. using a plate reader (SpectraMax; Molecular Devices, LLC, CA, USA) to monitor the change in absorbance as a function of time; data were normalized to a per-second scale as described in Green et al., Science (2016) 351(6273):617-21. Data are presented as means (standard deviation [SD]) in text or as mean±standard error of the mean (SEM) in figures; half maximal effective concentration (EC₅₀) values (and 95% confidence intervals [CIs]) were calculated using a four-parameter-fit model (GraphPad Prism, GraphPad Software Inc., CA, USA).

### Myocardial Force Generation Studies

The ability of Compound I to increase myocardial force generation at a given Ca₂₊ concentration was evaluated using LA (n=6) and LV (n=6) skinned muscle fibers prepared from three different Yucatan mini-pig hearts. In short, as previously described, 3 hearts were harvested, rinsed and shipped in cold cardioplegia solution (Custodiol® HTK; Essential Pharmaceuticals, LLC, NC, USA). Upon receipt, LV (papillary) and LA muscle fibers were dissected at 4° C. in a high-relaxing solution (100 mM BES, 10 mM EGTA, 6.57 mM MgCl2, 10 mM creatine phosphate, 6.22 mM ATP, 41.89 mM Kprop, 2.5 μM pepstatin, 1 μM leupeptin, 50 μM PMSF, 5 mM NaN3, pH 7.0). Fiber bundles were cut and skinned (in a high-relaxing solution containing 1% Triton X-100), fitted with aluminum foil t-clips, and mounted on a mechanics apparatus (Aurora Scientific Inc., ON, Canada). Sarcomere length was set to 2.0 μm. Steady-state isometric tension and stiffness (via a 3% stretch over 250 ms) were measured at increasing concentrations of Ca²⁺ (pCa 8.0 to 4.5, adjusted to maintain 180 mM ionic strength) twice, first in the absence (control, 1% DMSO) and then in the presence of Compound I (3 μM, 1% DMSO). In all cases, tension values were normalized to the control maximum isometric tension (at pCa 4.5). Active and passive stiffness were calculated by measuring the slope of the early (Ca²⁺ dependent) and late phases, respectively, of the tension response to the brief 3% stretches. Data are presented as means (SD) in text or as mean±SEM in figures; EC₅₀ values (and 95% CIs) were calculated using a four-parameter-fit model (GraphPad Prism, GraphPad Software Inc., CA, USA).

### Results

Compound I increased ATPase activity and calcium sensitivity in LV and LA myofibrils/muscle fibers.

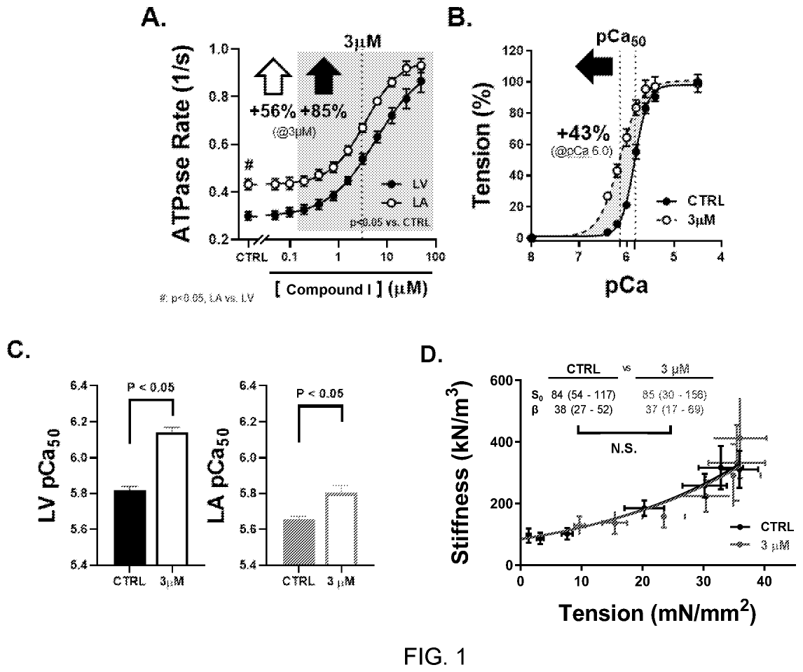

Compound I was associated with a dose-dependent increase in sarcomere activity (ATPase turnover rate) in both ventricular (half maximal active concentration [AC₅₀]: 6.0 μM; 95% confidence interval [CI]: 3.7-27.5) and atrial (AC₅₀: 3.6 μM; 95% CI: 2.7-5.0) myofibrils, achieving increases (±standard deviation [SD]) of 3.0-fold (±0.3) and 2.3-fold (±0.3), respectively, at 50 μM (FIG. 1, Panel A). Compound I activated cardiac (human) 51 myosin (1.4-fold [9] increase in ATPase rate at 3 μM), but not skeletal or smooth muscle isoforms (data not shown). In skinned fibers, Compound I (at 3 μM) shifted the tension-pCa²⁺ relationships leftwards (i.e. generated greater tension at a given Ca²⁺ concentration), increasing the Ca²⁺ sensitivity (pCa₅₀, [±SD] p<0.05 vs pre-treatment values) of both ventricular fibers (from 5.8 [±0.04] to 6.1 [±0.07], FIG. 1, Panels B and C) and atrial fibers (from 5.7 [±0.05] to 5.8 [±0.10], FIG. 1, Panel C), without altering passive stiffness (FIG. 1, Panel D) or maximal force-generation capability (data not shown).

In summary, Compound I at 3 μM increases ATPase by 56% in LA and 85% in LV porcine myofibrils; shifts calcium sensitivity to the left, increasing LV tension by 43% at pCa 6.0; and significantly increases calcium sensitivity in both LV and LA porcine myofibrils. These data show that Compound I increases ATPase activity and calcium sensitivity in both the LA and LV, resulting in increased contractile force.

### Example 2: In Vivo Functional Study of the Effect of Compound I on Left Atrial Function

This example evaluates the ability of Compound I to improve myocardial performance in vivo in the presence of chronic LV dysfunction/remodeling.

### Materials and Methods

Seven male beagle dogs underwent a modified serial coronary microembolization protocol to produce chronic LV dysfunction and HF (Geist et al., J Pharmacol Toxicol Methods (2019) 99:106595), as determined by both LV remodeling and decreases in LV ejection fraction (LVEF). A subset of animals (n=5) were also surgically implanted with a radio-telemetry transmitter (TL11M3-D70-PCTP; Data Sciences Int., MN, USA) to provide systemic arterial blood and LV pressures. The microembolization and instrumentation techniques employed have been previously validated (Hartman et al., JACC Basic Transl Sci. (2018) 3(5):625-38).

The effects of Compound I (at 2-3 mg/kg oral tablet; n=14) on LV/LA function and geometry, as well as systemic/ventricular haemodynamics, were examined by echocardiography in conscious, lightly sedated animals (butorphanol 0.25-0.5 mg/kg intravenous) before dosing (i.e., baseline) as well at 5 hours post-treatment.

In these experiments, 2D and 2D-guided M-mode echocardiographic (CX50; Philips Medical System, MA, USA) recordings of LV dimension, LA dimension (LAd) and aortic dimension (Aod), as well as LV volume estimates (Simpson's and Teichholz's methods), were obtained at end-systole/diastole in both short-axis (papillary-level) and/or apical/parasternal long-axis views. From these measurements, LV stroke volume (LVSV), cardiac output (CO), LV fractional shortening (LVFS), LV fractional area of shortening and LVEF, as well as the LAd/Aod ratio, were calculated. LV outflow tract (LVOT) blood velocity (via Doppler) was measured and the LVOT velocity−time integral (LVOT−VTI) calculated. In addition, maximal (end-systolic, LAₘₐₓ) and minimal (end-diastolic, LAₘᵢₙ) LA volumes were measured using the bi-plane method; both LA emptying fraction (LAEF=100×[LAₘₐₓ−LAₘᵢₙ]/LAₘₐₓ) and LA functional index (LAFI=[LAEF×LVOT−VTI]/LAₘₐₓ index) were calculated (Thomas et al., Eur J Echocardiogr (2008) 9(3):356-362). Diastolic trans-mitral peak flow velocities (E and A), mitral-annulus tissue velocities (e′, s′ and a′), and their ratios during early filling (E/e′), were recorded/used as indices of diastolic performance. In all cases, atrial and ventricular indexed volumes were calculated by normalizing to the estimated body surface area (0.101×[body weight in kg]×⅔), while reported data were derived by averaging at least three cardiac cycles. Finally, haemodynamic signals were digitally acquired (1000 Hz) and recorded continuously with a data acquisition/analysis system (IOX; EMKA Technologies). Heart rate (HR) and end-systolic and end-diastolic pressures, as well as the peak rates of pressure rise and decline (dP/dtₘₐₓ and dP/dtₘᵢₙ, respectively), the contractility index (dP/dt/P at dP/dtₘₐₓ), and the time constant of myocardial relaxation (tau_{1/2}, time for 50% decay from dP/dtₘᵢₙ) were derived from the LV pressure signal. Systolic, diastolic and mean systemic blood pressures, as well as pulse pressures, were derived from the aortic pressure signal. Haemodynamic data were reported as the average over at least 1 minute (at steady state). In vivo data are presented as means (SD) in the text or as mean±SEM in the figures; mean differences between pre- and post-treatment values were evaluated via a two-tailed paired t-test with a significance level of 0.05 set a priori (GraphPad Prism, GraphPad Software Inc., CA, USA).

### Results

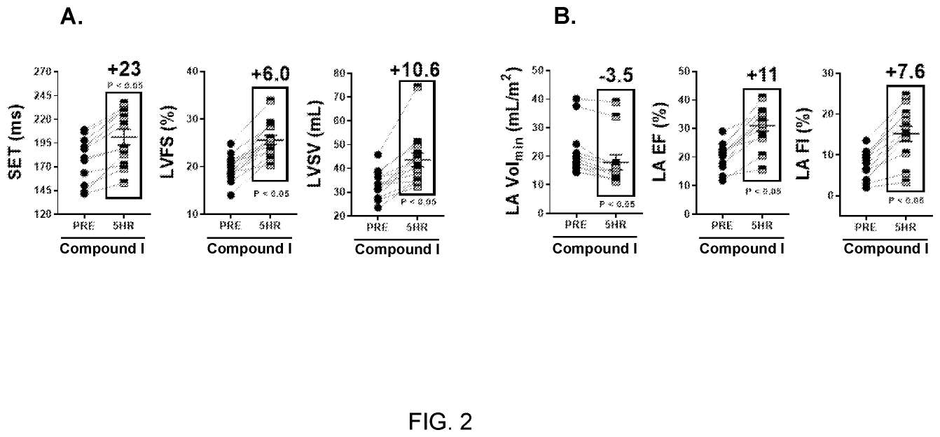

In dogs with microembolization-induced heart failure, acute treatment with Compound I improved LVEF [±SD] (41 [5]% to 51 [6]%; p<0.05), LVFS (19.6 [2.7]% to 25.6 [3.6]%; p<0.05) and peak LV global circumferential strain (LVGCS: −13.5 [4.4]% to −17.3 [4.4]%; p<0.05), leading to increases in both LVSV (33.0 [5.9] mL vs 43.6 [10.7] mL; p<0.05) (FIG. 2, Panel A) and cardiac output (Table 4). Additionally, Compound I prolonged SET (178 [24] ms vs 201 [29] ms; p<0.05) (FIG. 2, Panel A), but had negligible effects on LV end-diastolic dimensions, derived indices of ventricular filling or LV filling pressures (Table 4). In a subset of dogs instrumented for systemic/LV haemodynamics (via telemetry), Compound I had no effect on systemic pressures (±SD), such as systolic blood pressure (110 [10] vs 119 [10] mmHg) or LV end-diastolic pressures (18 [2] to 16 [4] mmHg), despite a slight reduction in heart rate (108 [45] to 99 [50] bpm; p<0.05).

Compound I also reduced LA volumes, particularly at end-diastole (LA minimal volume index [LAₘᵢₙVi]: 21.2 [8.3] mL/m² vs 17.9 [9.0] mL/m²; p<0.05), improving both the LA emptying fraction (LAEF: 20.4 [4.4]% vs 31.1 [6.9]%; p<0.05) and the LA function index (Thomas et al., Eur J Echocardiogr (2008) 9(3):356-62) (LAFI: 7.7 [3.3]% vs 15.2 [6.5]%; p<0.05) (FIG. 2, Panel B and Table 4).

TABLE 4
Cardiac and haemodynamic effects of acute Compound I (2-3 mg/kg
orally) administration in dogs with induced heart failure

| | | |
|---|---|---|
| Parameter, mean (±SD) | Baseline | Compound I |

Measures of LV systolic function

| | | | | |
|---|---|---|---|---|
| LVSV, mL | 33.0 | (±5.9) | 43.6 | (±10.7)* |
| CO, L/min | 3.4 | (±0.9) | 4.1 | (±1.6)* |
| LVEF, % | 41 | (±5) | 51 | (±6)* |
| LVFS, % | 19.6 | (±2.7) | 25.6 | (±3.6)* |
| SET, ms | 178 | (±24) | 200 | (±30)* |
| LVGCS, % | −13.5 | (±4.4) | −17.3 | (±4.4)* |

LV dimensions and volumes

| | | | | |
|---|---|---|---|---|
| LVEDD, cm | 4.3 | (±0.2) | 4.3 | (±0.3) |
| LVESD, cm | 3.4 | (±0.2) | 3.2 | (±0.2)* |

Relaxation/diastolic function

| | | | | |
|---|---|---|---|---|
| e′, cm/s | 7.4 | (±1.0) | 7.3 | (±1.1) |
| E/e′, n/u | 9.7 | (±1.9) | 10.2 | (±1.2) |
| E, cm/s | 71 | (±12) | 74 | (±40) |
| A, cm/s | 41 | (±6) | 46 | (±8) |
| E/A | 1.6 | (±0.2) | 1.6 | (±0.3) |
| LVEDP, mmHg | 18 | (±2) | 16 | (±4) |
| a′, cm/s | 4.0 | (±1.2) | 5.4 | (±1.3)* |

Left atrium volume and function

| | | | | |
|---|---|---|---|---|
| LAEF, % | 20.4 | (±4.4) | 31.1 | (±6.9)* |
| LAₘₐₓ Vol. index, mL/m² | 26.7 | (±9.7) | 25.1 | (±9.7)* |
| LAₘᵢₙ Vol. index, mL/m² | 21.2 | (±8.3) | 17.9 | (±9.0)* |
| LAFI | 7.7 | (±3.3) | 15.2 | (±6.5)* |

Vital signs (supine)

| | | | | |
|---|---|---|---|---|
| HR, bpm | 108 | (±45) | 99 | (±50)* |
| SBP, mmHg | 110 | (±10) | 119 | (±10) |
| DBP, mmHg | 66 | (±10) | 72 | (±13) |

Data are means (±SD).
A, late peak wave velocity from mitral inflow Doppler; a′, late peak mitral annular velocity in diastole; bpm, beats per minute; CO, cardiac output (estimated); DBP, diastolic blood pressure; E early peak wave velocity from diastolic trans-mitral Doppler flow, respectively; e′ late peak mitral annular velocity in diastole, respectively; HF, heart failure; HR, heart rate; LAEF, left atrial emptying fraction; LAFI, left atrial functional index; LAₘₐₓ and LAₘᵢₙ, indexed left atrial maximum (end-systolic) and minimum (end-diastolic) volumes, respectively; LVEDD and LVESD, left ventricular end-diastolic and end-systolic diameters, respectively; LVEDP, left ventricular end-diastolic pressure; LVEF, left ventricular ejection fraction; LVFS, left ventricular fractional shortening; LVGCS, left ventricular global circumferential strain; LVSV, left ventricular stroke volume; SBP, systolic blood pressure; SD, standard deviation; SET, systolic ejection time.
*p < 0.05.

### Example 3: In Vivo Functional Study of the Effect of Compound I on Atrial Fibrillation Inducibility

This example evaluates the effects of Compound I on left atrial function and size, and studies its potential implications to alter atrial fibrillation substrates.

### Materials and Methods

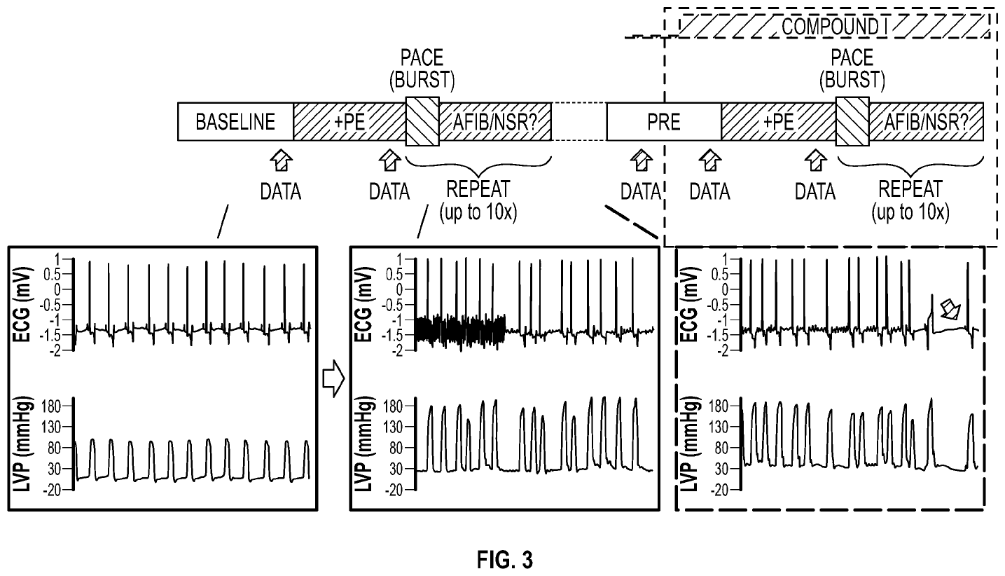

The effects of Compound I on AF inducibility and LA size and function were examined by echocardiography (ECHO) and electrocardiogram (ECG) in beagle dogs with phenylephrine (PE) on board, and compared to the effects of PE alone. The experimental design is shown in FIG. 3.

Beagle dogs (n=8) were acutely studied under isoflurane anesthesia. A subset of the animals (n=3) had chronically induced left ventricular dysfunction (EF<40%). Animals were assigned to either vehicle or Compound I. Anesthesia procedures were performed, and a percutaneous introducer (under strict aseptic conditions) placed into the jugular vein to insert a catheter into the right atrium or coronary sinus. Once instrumented and stable, the first anesthetized ECHO was performed, and blood was drawn for analysis.

Following the first ECHO and blood draw, once a steady baseline had been achieved, the AF inducibility protocol was performed, consisting of five-to-ten×10-second burst pulses at 33 Hz. After each pulse, the seconds that AF persisted were recorded. AF was identified by (1) a presence of irregular rapid ventricular response, (2) an absence of a P wave, and (3) a presence of low-frequency irregular oscillations (f waves). If AF spontaneously converted to sinus rhythm after less than 10 minutes, the next pulse was delivered. Once regular sinus rhythm returned, each 10-second burst was separated by roughly the same amount of time as the duration of the previous AF. If no AF was present, then each burst was separated by ˜10-30 seconds after the previous burst pace was completed. If AF persisted for over 20 minutes, the inducibility protocol was halted and the duration of AF recorded. If at any time, AF did not spontaneously convert before the animal was due to recover from anesthesia, medical conversion could be attempted. If the five-to-ten×10-second burst pulses at 33 Hz did not produce consistent AF, then the right atria could be stimulated at various frequencies (˜10-33 Hz) and for longer durations (˜10 second to 15 minutes).

Subsequently, PE (0.5-20 μg/kg/min) was administered at a fixed rate. In the animals, phenylephrine (PE) induced elevated systemic/left-ventricular pressures (e.g., SBP: 38±7%, 88.6±2.7 to 122.4±6.9 mmHg, P<0.05), increasing left-atrial dimensions (e.g., LAₘᵢₙ: +28±3%, 22.9±2.0 to 29.3±2.5 mL/m², P<0.05) and creating a substrate for the induction of AF via brief (10s) right-atrial burst-pacing bouts.

After 5-15 minutes (or sufficient time for steady state), a second anesthetized ECHO was performed. Once completed, the AF inducibility protocol (as described above) was performed. All observations (as visualized via ECG recordings) of AF were documented, including the duration that the AF was sustained. PE was then turned off and the PE-mediated hemodynamic effects were allowed to recover (i.e., washout). Once sinus rhythm was re-established (cardioversion was used if AF persisted for more than 30 minutes), a third anesthetized ECHO was performed. Next, either Compound I or vehicle was administered IV via a suitable vein. Treatment consisted of a bolus and an IV infusion (titrated to match LV end systolic pressure or peak LVP as when PE was administered alone and targeting for the exact same dose). After 10 minutes of infusion, a fourth anesthetized ECHO was performed. Next, a PE infusion (the same fixed rate as above was used) was started in combination with Compound I or vehicle. Upon observation of stable hemodynamics (˜10 minutes), blood was drawn for analysis, and the final ECHO and an AF inducibility protocol were performed. Following completion of a successful AF inducibility protocol and return to normal sinus rhythm, the animals were allowed to recover.

### Results

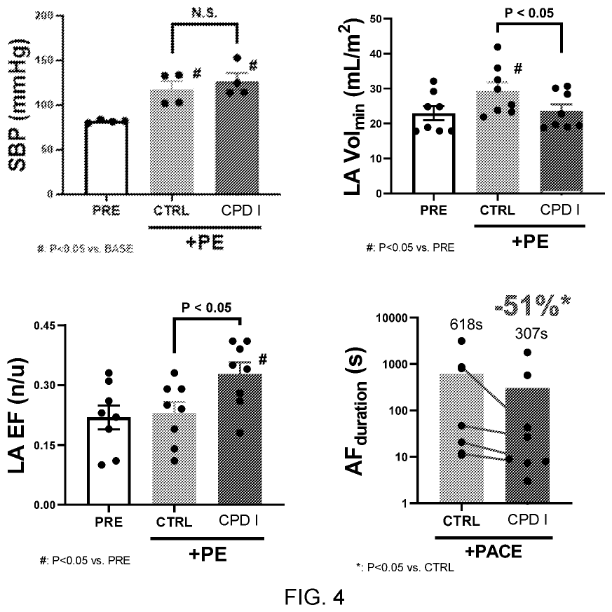

Acute Compound I administration (0.3-0.4 mg/kg IV bolus, with 0.3/0.4 mg/kg/hr IV infusion) prolonged systolic ejection time (SET: +10±3%, P<0.05), increasing left-ventricular stroke volume (SV: 16±5%, P<0.05) and fractional shortening (FS: 13±3%, P<0.05); systemic pressures were preserved under Compound I (SBP: 135.7±6.2 mmHg). However, Compound I decreased left-atrial size (LA Volmin), increasing the atrial emptying fraction (LA EF) and decreasing AF inducibility (e.g., AF duration) (FIG. 4). In summary, Compound I significantly blunted AF inducibility while decreasing left atrial size, compared to control conditions.

### Example 4: In Vivo Functional Study of the Effect of Dobutamine on Left Atrial Function and Atrial Fibrillation Inducibility

This example evaluates the effects of the inotropic agent dobutamine on left atrial function and size, as well as atrial fibrillation inducibility.

### Materials and Methods

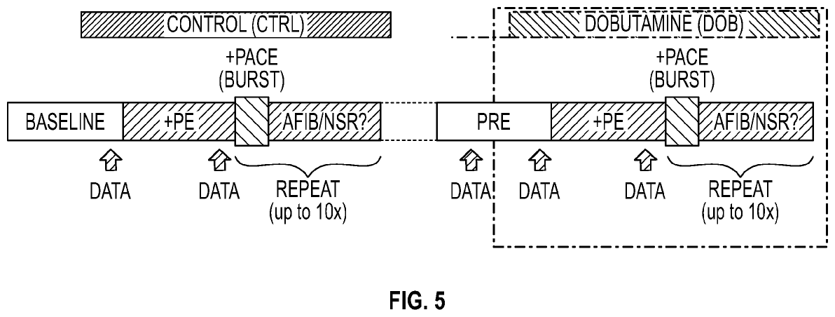

The effects of dobutamine on AF inducibility and LA size and function were examined by echocardiography (ECHO) and electrocardiogram (ECG) in beagle dogs with phenylephrine (PE) on board, and compared to the effects of PE alone. The beagle dogs (without left ventricular dysfunction) were assessed for left atrial function and size and for atrial fibrillation inducibility according to the protocols described in Example 3 (experimental design shown in FIG. 5), allowing for the effects of dobutamine to be compared to those of Compound I as shown in Example 3.

### Results

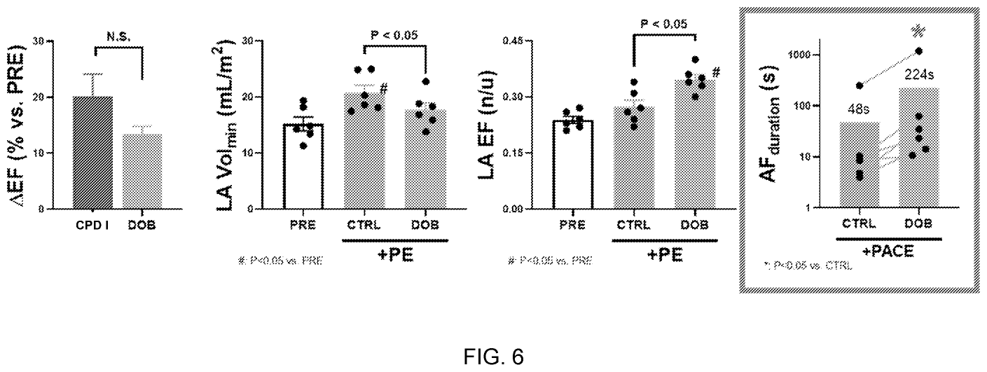

Dobutamine is an inotropic agent that increases LV contractility through a mechanism of action different from that of Compound I. The effects of dobumatine administration (1-10 μg/kg/hr infusion) were compared to those described in Example 3 for Compound I. Both agents were shown to increase LV contractility (ΔEF; FIG. 6, leftmost panel). However, unlike Compound I, dobutamine did not reduce AF duration, and in fact increased it (FIG. 6, rightmost panel). These results demonstrate that the ability of Compound I to reduce AF duration is due to its unique mechanism, and is not a general result of increasing LV contractility.

### Example 5: Randomized, Double-Blind, Placebo-Controlled, Two-Part, Adaptive Design Study of the Effect of Compound I on Left Atrial Dimensions and Function in Patients with Stable HFrEF

This example describes a study to establish the effect on left atrial dimensions and function of single- and multiple-ascending oral doses of Compound I in ambulatory patients with stable heart failure with reduced ejection fraction (HFrEF). Key eligibility criteria included stable HFrEF of ischemic or nonischemic origin, treated with guideline-directed medical therapy (EF initial requirement during Screening was 20 to 45%, and was later changed by amendment to 15 to 35%). Subjects with active ischemia or severe or valvular heart disease were excluded.

### Materials and Methods

### Study Design

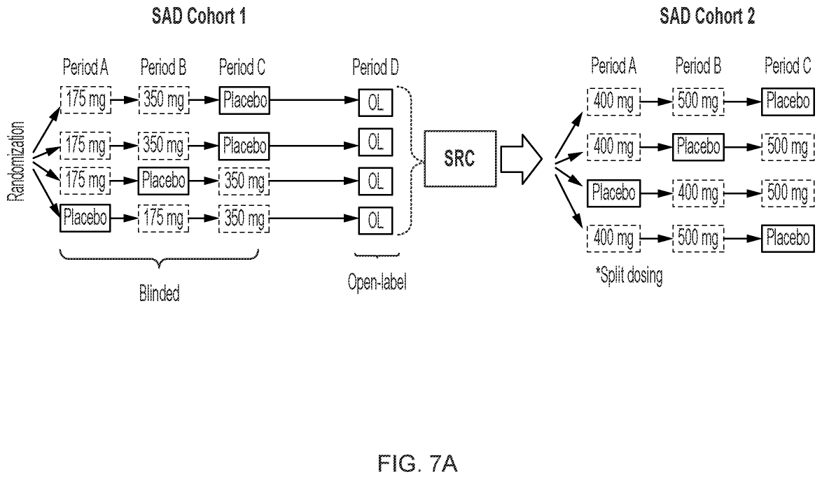

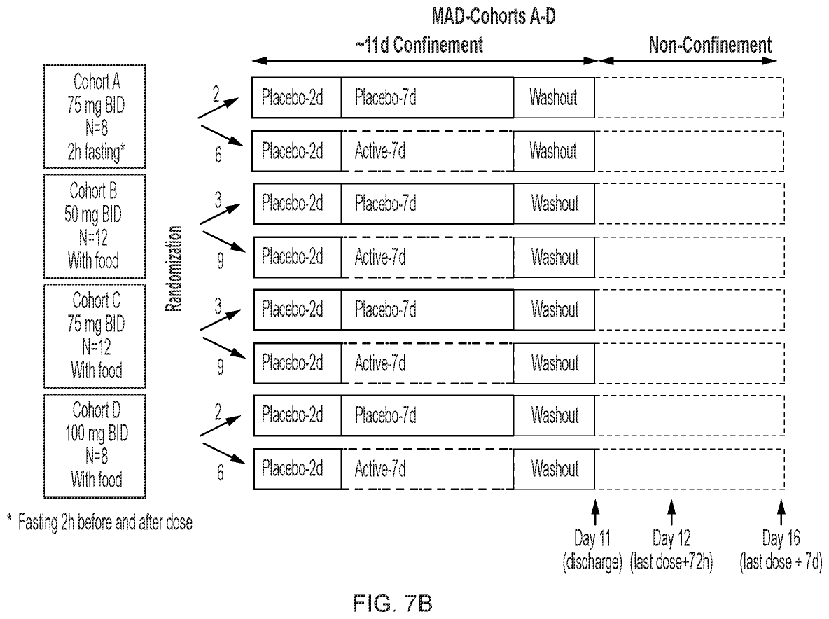

Part 1 of this two-part study evaluated single-ascending doses (SAD) of Compound I, and Part 2 evaluated multiple-ascending doses (MAD) of Compound I (FIGS. 7A and 7B).

The clinical trial enrolled patients who were 18-80 years of age with a clinical diagnosis of stable chronic heart failure with an LV ejection fraction (LVEF) on echocardiography of 45% or less (subsequently amended to ≤35%), treated with guideline-directed medical therapy, and with good quality echocardiogram images. Patients were excluded if they had renal impairment (estimated glomerular filtration rate <30 mL/min/1.73 m²), if their screening cTnI was elevated (value measured at the central laboratory using Abbott Architect assay >0.15 ng/mL, with upper limit of normal of 0.03 ng/mL), if they had been admitted to hospital for heart failure or had an acute coronary syndrome or intervention in the previous 90 days, or had uncorrected severe valvular disease. Patients with current or recent AF were also excluded. Detailed inclusion and exclusion criteria are shown below.

### Inclusion Criteria

1. Men or women 18-80 years of age at the screening visit
2. BMI 18-40 kg/m², inclusive
3. Sinus rhythm or stable atrial pacing with mean resting HR 50-95 beats per minute (bpm), inclusive (patient will be ineligible to dose if, on Day 1, the pre-dose HR measurement is ≥95 bpm. HR is the mean of three measurements taken 1 minute apart. A single measurement would not make a patient ineligible)
4. Has stable, chronic HFrEF of moderate severity, as defined by all of the following
   For the first three patients in each cohort of the multiple-dose trial testing a new (higher) daily dose: documented LVEF 25-35% during screening (as confirmed by ECHO Central Laboratory)
   For other patients in the multiple-dose trial cohorts (and all patients in single-ascending dose trial): documented LVEF 15-35% during screening (as confirmed by ECHO Central Laboratory)
      LVEF must be confirmed with second screening ECHO to be performed at least 7 days after initial screening ECHO. Results of both must meet inclusion criteria and must be received from core laboratory prior to dosing. In the event of extended screening windows due to SRC reviews, effort should be made to ensure second ECHO is near planned time of randomization
   Chronic medication for the treatment of heart failure consistent with current guidelines, which has been given at stable doses for ≥2 weeks with no plan to modify during the study. This includes treatment with at least one of the following, unless not tolerated or contraindicated: beta-blocker, angiotensin converting enzyme (ACE) inhibitor/angiotensin receptor blocker (ARB)/angiotensin receptor neprilysin inhibitor (ARNI)

### Exclusion Criteria

1. Inadequate echocardiographic acoustic windows
2. Any of the following ECG abnormalities: (a) QTcF>480 ms (Fridericia's correction not attributable to pacing or prolonged QRS duration, average of triplicate screening ECGs) or (b) second-degree atrioventricular block type II or higher in a patient who has no pacemaker
3. Hypersensitivity to Compound I or any of the components of the Compound I formulation
4. Active infection, as indicated clinically and as determined by the investigator
5. History of malignancy of any type within 5 years prior to screening, with the exception of the following surgically excised cancers occurring more than 2 years prior to screening: in situ cervical cancer, non-melanomatous skin cancers, ductal carcinoma in situ and non-metastatic prostate cancer
6. Positive serologic test at screening for infection with human immunodeficiency virus (HIV), hepatitis C virus (HCV) or hepatitis B virus (HBV)
7. Hepatic impairment (defined as alanine aminotransferase (ALT)/aspartate aminotransferase (AST)>3 times ULN and/or total bilirubin (TBL)>2 times ULN)
8. Severe renal insufficiency (defined as current estimated glomerular filtration rate [eGFR] <30 mL/min/1.73 m² by simplified Modification of Diet in Renal Disease equation [sMDRD])
9. Serum potassium <3.5 or >5.5 mEq/L
10. Any persistent out-of-range safety laboratory parameters (chemistry, haematology, urinalysis) considered by the investigator and medical monitor to be clinically significant
11. History or evidence of any other clinically significant disorder, condition or disease (including substance abuse) that, in the opinion of the investigator would pose a risk to patient safety or interfere with the study evaluation, procedures or completion, or lead to premature withdrawal from the study
12. Participated in a clinical trial in which the patient received any investigational drug (or is currently using an investigational device) within 30 days prior to screening, or at least 5 times the respective elimination half-life (whichever is longer)
13. Previous participation in a clinical trial with Compound I, with the exception that patients who participated or were screen failures in one part of this trial may participate in the other part, i.e. patients may enroll in Part 1 (single-ascending dose trial) followed by Part 2 (multiple-dose trial) or Part 2 (multiple-dose trial) followed by Part 1 (single-ascending dose trial), with the following caveats
   If the patient has an ongoing AE, has had an SAE or has met any stopping criteria, the investigator should contact the sponsor prior to enrolling the patient in a subsequent cohort
   Patients must have at least 1 week of washout after the end of the multiple-dose trial dosing prior to the single-ascending dose trial dosing, or at the end of single-ascending dose trial dosing prior to the multiple-dose trial dosing
   Patients do not need to rescreen if the multiple-dose trial screening occurred within 12 weeks of the first single-ascending dose trial dosing, or if the single-ascending dose trial screening occurred within 12 weeks of the first multiple-dose trial dosing. Investigators should verify that patients are clinically stable and no exclusions have occurred during the interim; if >12 weeks have elapsed or if there is clinical instability, then patients should be rescreened
14. At screening, symptomatic hypotension, or systolic BP>170 mmHg or <90 mmHg, or diastolic BP>95 mmHg, or HR<50 bpm. HR and BP will be the mean of three measurements taken at least 1 minute apart
15. Current angina pectoris
16. Recent (<90 days) acute coronary syndrome
17. Coronary revascularization (percutaneous coronary intervention [PCI] or coronary artery bypass graft [CABG]) within the prior 3 months
18. Recent (<90 days) hospitalization for heart failure, use of chronic IV inotropic therapy or other cardiovascular event (e.g. cerebrovascular accident)
19. Uncorrected severe valvular disease
20. Elevated troponin I (>0.15 ng/mL) at screening, based on Central Laboratory assessments. Note: Central Laboratory troponin I assay ULN is 0.03 ng/mL
21. Presence of disqualifying cardiac rhythms that would preclude study ECG or echocardiographic assessments, including: (a) current AF, (b) recent (<2 weeks) persistent AF or (c) frequent premature ventricular contractions. Note: patients with active cardiac resynchronization therapy (CRT) or pacemaker (PM) are eligible if initiated at least 2 months prior to the study, with no plan to change CRT or PM settings during the study
22. A life expectancy of <6 months

Part 1 (SAD Cohorts)

In the single-ascending dose trial, single fasting doses of Compound I 175-550 mg or placebo were evaluated in a crossover manner in 12 patients over two sequential cohorts (1 and 2), with intervals between singles doses ranging from 3 days (subsequently amended to 5) to 14 days. In Cohort 1, eight patients were enrolled and all received placebo and Compound I 175 mg and 350 mg (in random sequence and in a blinded fashion) during three periods, A-C. Six patients elected to continue into a fourth optional open label period D. Compound I doses administered in the open-label period included: 350 mg (n=1); 450 mg (divided into two administrations; n=1); 525 mg (n=2); and 550 mg (divided into two administrations; n=2). In Cohort 2, four patients were enrolled, and all received placebo and Compound I 400 mg and 500 mg in random sequence (both active 400 mg and 500 mg doses were divided into two administrations).

For each treatment period, pre-dose assessments followed by administration of single dose were performed on Day 1 in the morning. The patient underwent serial pharmacokinetic (PK), pharmacodynamic (PD [trans-thoracic echocardiography or TTE]), ECG and safety laboratory assessments through Day 1 (until the evening) as well as on Day 2. Patients were discharged on Day 3 morning and returned to the clinic on Day 4 for a final PK assessment, and evaluation of adverse events (AEs). After completion of all treatment periods, a 7-day follow-up visit was completed at the site.

Part 2 (MAD Cohorts)

This was a randomized, parallel-group, DB, placebo-controlled, adaptive design, sequential ascending (oral) multiple-dose study in stable patients with heart failure. Four MAD Cohorts (A, B, C, D) were enrolled (FIG. 7B). An SRC reviewed results from each cohort and determined the dose and confirmed initial sample size for the subsequent cohort. Additionally, the first 3 patients in each cohort had LVEF>25%; the SRC reviewed preliminary safety data from these patients and decided whether to open cohort enrollment to patients with LVEF<25%.

In Cohort A, Compound I 75 mg twice daily (BID) or matching placebo was administered after a 2-hour fast, and food was not allowed for the following 2 hours. In Cohorts B, C and D, patients received Compound I 50, 75 and 100 mg BID, respectively, with food (Table 5).

TABLE 5
Multiple-dose trial dosing cohorts

| | | Number of patients |
|---|---|---|
| | | treated with |
| | Dose | Compound I* |
| Cohort A (n = 8) | 75 mg BID | 6 |
| Cohort B (n = 12) | 50 mg BID | 9 |
| Cohort C (n = 12) | 75 mg BID | 9 |
| Cohort D (n = 8) | 100 mg BID | 6 |

Patients were admitted to a clinical research unit for 11 days and underwent 3 consecutive study periods: (1) an initial single-blind placebo run-in period of 2 days (Days 1-2); (2) a randomized (1:3) double-blind treatment period in which patients received 7 days of placebo or Compound I, administered orally twice daily (from Day 3 until Day 9); (3) a follow-up period with patients discharged from the unit on D11, and a final follow-up clinic visit conducted on Day 16. During the 11-day confinement, patients were under continuous supervision. Occasionally, patients who had an ICD implanted, were allowed not to be confined but were still closely monitored, returning frequently to the clinical research unit and with each intake of double-blind treatment supervised by a healthcare professional.

Patients were dosed twice daily (every 12 hours). Doses could occur ±2 hours from scheduled dosing times as long as doses were separated by at least 10 hours and by no more than 14 hours. The exception to the twice daily dosing was on Day 9 (last dose of randomized DB study drug treatment). On Day 9, a single morning dose was administered.

Before each dosing event, all available safety data from the previous days was reviewed (for non-confined patients, if a home health nurse was utilized, the nurse and site were in daily communication to ensure safety). Dosing of DB treatment took place at approximately the same time each day.

Compound I was supplied as an oral tablet that was blistered and carded. Placebo tablets were provided and presented in matching form. All clinical trial material was manufactured, packaged, labelled, and distributed by Sanofi, Inc (Montpellier, France). Each blister card contained either 25 mg tablets, 100 mg tablets or placebo tablets. There were no mixed-strength blister cards utilized. Each blister card was labelled as required by local regulations and in a manner to allow a local unblinded pharmacist to prepare each dose during the double-blind treatment period. Other than the unblinded pharmacist, other site study personnel remained blinded to the treatment assignment.

During the study, multiple evaluations were performed that included: serial TTE assessments (11-14 TTEs per patient on Days 1, 2, 3, 4, 7, 9, 10 and 11); PK sampling (PK sample collected concomitantly with every post-randomization echocardiogram); ECGs (on Days 2, 3, 4, 7, 9, 10, 11 and 16); troponin (collected concomitantly with every post-randomization ECG); and safety laboratory assessments. Confined patients underwent continuous telemetry. Holter monitoring was performed in all patients at baseline (Days 1-2) and at the end of double-blind treatment (Days 7-9). Vital signs were collected daily.

In addition to the central assessments, local assessments of 12-lead ECGs, TTEs, safety laboratory results and troponin were performed by sites for real-time safety monitoring and patient management. Clinicians were instructed by protocol to implement immediate dose modification (i.e. administer a lower dose) in the case of PD effects on TTEs considered excessive (based on local TTEs); such as systolic ejection time prolongation >75 msec on two sequential TTEs or >110 msec on a single TTE as compared to baseline (Day 3, pre-dose), or >50% relative increase in two contractility parameters in two successive TTEs. Dosing was also to be discontinued in the case of drug-related coronary ischaemia, drug-related suspected unexpected serious adverse reaction, liver injury or clinically significant and persistent changes in vital signs or arrhythmias or HR-corrected QT interval using Fridericia's method (QTcF)>500 msec (not attributable to pacing or prolonged QRS duration).

### Study Treatment

In Part 1 (SAD), study patients received separate ascending doses of Compound I (2 to 3 doses) and a single dose of matching placebo. In Part 2 (MAD), study patients received single-blind placebo BID for Days 1 and 2 and then received DB treatment (either placebo or Compound I) for 7 days (Days 3 through 9). In Cohorts A, B, C, and D, on Day 9 patients received a single dose of placebo or Compound I in the morning for serial PK/PD assessments, while on Days 3 through 8 patients in these cohorts received placebo or Compound I BID.

Compound I drug substance was as described in Example 1 above and was provided as 5, 25, or 100 mg tablets. Placebo tablets were provided as matching tablets. The tablets were blistered and then carded. Each blister card contained only 5 mg, only 25 mg, only 100 mg, or only placebo. The blister cards were packaged into “Kit Boxes.”

### Study Medication, Administration, and Schedule

Study medication consisted of Compound I 5 mg tablets, 25 mg tablets, 100 mg tablets, or matching placebo tablets. In Part 1 (SAD), Compound I or placebo was administered after an overnight fast (at least 6 hours), while in Part 2 (MAD), Compound I was administered after a 2 hour fast (Cohort A) or with food (Cohorts B, C, and D). The dose was ingested with a minimum of 240 mL of water, but more water was ingested as needed. The entire dose was administered over a period of up to 15 minutes. The time of dose used to determine future assessments was the time the last tablet was taken. In the cohorts for Part 2 (MAD), a BID regimen was used.

In Part 1 (SAD), patients fasted overnight (approximately 6 hours) through 4 hours postdose. With the exception of the water consumed with the dose, water could be ingested until approximately 1 hour prior to dosing and approximately 1 hour after dosing. If doses were split, subjects fasted 6 hours prior to the first half-dose. A light, low-fat snack could be consumed 2 hours after the first half-dose and a fast continued through 2 hours after the second half-dose.

In Part 2 (MAD), Cohort A patients fasted for 2 hours before and 2 hours after dosing. For example, if morning dosing occurred at 8 AM, patients could have a snack at 6 AM and a full breakfast at 10 AM. If afternoon dosing occurred at 8 PM, patients could have dinner at 6 PM and a snack at 10 PM. These times could be adjusted based on local scheduling preferences, but doses were separated by at least 10.5 hours. Cohort B, C, and D patients ingested food with each dose.

### Management of an Exaggerated Pharmacological Effect and Overdose

Based on the nonclinical pharmacological characteristics, exaggerated effects of Compound I could lead to myocardial ischemia. The duration of effect would follow the PK profile of Compound I with a Tₘₐₓ of 4 to 6 hours and a half-life of about 15 hours in healthy volunteers, but a slightly longer half-life in patients that received Compound I as part of Cohort 1 (20 to 25 hours). The clinical signs and symptoms, which could include chest pain, lightheadedness, diaphoresis, and ECG changes should start to abate over a short period of time. Any patient with signs and/or symptoms that might be secondary to cardiac ischemia was immediately evaluated by the physician for the possibility of cardiac ischemia and additional ECGs and serial troponins obtained as part of the evaluation as appropriate.

If evidence of cardiac ischemia was present, then the patient received standard therapy for ischemia as appropriate, including supplemental oxygen and nitrates. Caution in the administration of agents that increase HR was required, as Compound I may prolong the SET, which would result in decreasing the diastolic duration resulting in a decrease in diastolic ventricular filling. In addition, the exaggerated pharmacological effect could increase myocardial oxygen demand, so agents that might increase myocardial oxygen demand further were administered with caution.

Patients who received a greater dose than planned were supported as appropriate, such as described above if there is an exaggerated pharmacologic effect.

### Concomitant Therapy

During the study, the patients continued to ingest their medications for the treatment of their congestive heart failure and other medical conditions at the same doses and as close to the same times as usual, in order to maintain as best as possible similar preload and afterload conditions throughout the study to minimize confounding factors for the assessment of the effects of Compound I. In particular, if the patient was treated with diuretics, the time of administration of the diuretic relative to DB treatment was kept similar throughout the study. Times of administration of diuretics, if applicable, were collected. If the patient was not confined, the patient was instructed to maintain constant timing of daily administration of medications, including diuretics if applicable, and to record the time of administration.

All prescription and over-the-counter medications were reviewed by the investigator. Questions concerning enrollment or medications were discussed with the medical monitor. Over-the-counter medications could be taken at stable doses throughout the study (at investigator's discretion), and in amounts no greater than as directed per the label. All concomitant therapies (prescription or over-the-counter) were recorded. Other investigational therapies were discontinued at least 30 days prior to Screening or 5 half-lives (whichever is longer).

If the patient had an AE requiring treatment (including the ingestion of acetaminophen or ibuprofen), the medication was recorded; including time of the administration (start/stop), date, dose, and indication.

### Study Results

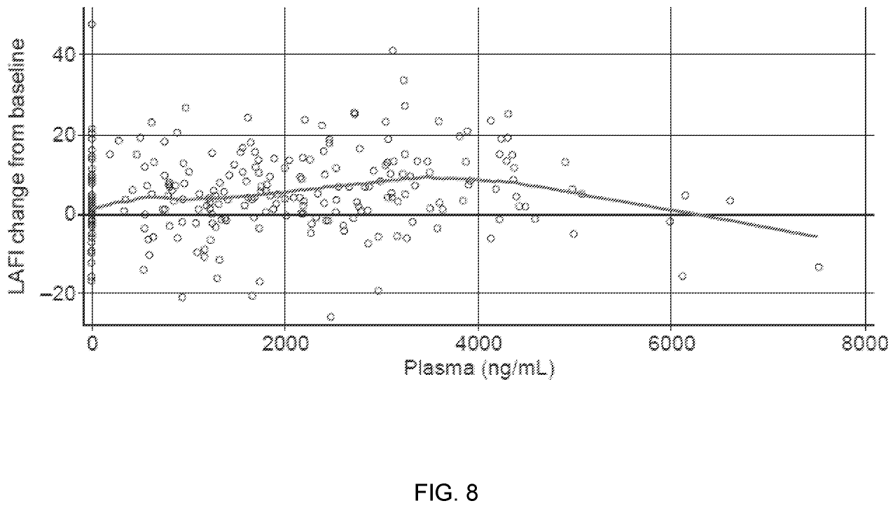

At 50 mg BID, Compound I achieved a steady-state concentration in the range of 2000 to <3500 ng/mL. Compound I significantly reduced LAₘᵢₙVi (−2.1 mL/m2 [p<0.01] and −2.4 mL/m2 [p<0.01] at medium and high concentrations, respectively), increased LAEF (+3.3% [p<0.05] and 3.6% [p<0.05] at medium and high concentrations, respectively), and improved LAFI (+6.1 [p<0.01] and +5.8 [p<0.01] at medium and high concentrations, respectively) (Table 6 and FIG. 8).

TABLE 6
Multiple-dose trial - change from baseline (placebo-corrected) in echocardiographic
variables according to Compound I plasma concentration ranges

| | Mean change (SE)^{b,c} by Compound I | | |
|---|---|---|---|
| | plasma concentrations group | | |
| Baseline^{a} | <2000 ng/mL | 2000-<3500 ng/mL | ≥3500 ng/mL |
| (n = 40) | (n = 30) | (n = 26) | (n = 13) |

Left atrial volume and function

| | | | | | | | | |
|---|---|---|---|---|---|---|---|---|
| LAEF (%) | 41 | (8) | 2.1 | (1.2) | 3.3* | (1.3) | 3.6* | (1.6) |
| LAₘₐₓVi (mL/m²) | 28 | (9) | −1.2 | (0.6) | −1.1 | (0.7) | −1.3 | (0.8) |
| LAₘᵢₙVi (mL/m²) | 17 | (7) | −1.8** | (0.6) | −2.1** | (0.6) | −2.4** | (0.7) |
| LAFI | 26 | (13) | 2.6 | (1.5) | 6.1** | (1.6) | 5.8** | (2.0) |
| MR jet area/LA | 8.7 | (10.5) | 0.3 | (1.2) | −0.6 | (1.3) | −4.2* | (1.6) |
| area ratio (%) | | | | | | | | |

A, late peak wave velocity from mitral inflow Doppler; bpm, beats per minute; DBP, diastolic blood pressure; e′, peak atrioventricular valve annular velocity in early diastole; E, early peak wave velocity from mitral inflow Doppler; IVRT, isovolumic relaxation time; LA, left atrial; LAEF, left atrial emptying fraction; LAFI, left atrial functional index; LAₘₐₓVi, left atrial maximum volume index; LAₘᵢₙVi, left atrial minimum volume index; LS, least-squares; LV, left ventricular; LVEDD, left ventricular end-diastolic diameter; LVEDVi, left ventricular end-diastolic volume index; LVEF, left ventricular ejection fraction; LVESD, left ventricular end systolic diameter; LVESVi, left ventricular end systolic volume index; LVFS, left ventricular fractional shortening; LVGCS, left ventricular global circumferential strain; LVGLS, left ventricular global longitudinal strain; LSVS, left ventricular stroke volume; MR, mitral regurgitation; SBP, systolic blood pressure; SD, standard deviation; SE, standard error; SET, systolic ejection time; TTE, transthoracic echocardiogram.
For the analysis, all assessments are included in the column corresponding to the Compound I concentration reached concomitantly to the assessments. As a result, 4 patients contributed to the lower (<2,000 ng/mL) Compound I concentration group only, 13 patients contributed to both the lower and medium (2,000-<3500 ng/mL) Compound I concentration groups, and 13 patients to all three Compound I concentration groups.
^{a}Absolute arithmetic mean values and SD for the baseline measurement for all Compound I-treated patients, excluding patients receiving placebo.
^{b}LS mean difference (SE) between each plasma concentration group (<2000 ng/mL, 2000-<3500 and ≥3500 ng/mL) and placebo (concentration = 0) in TTE parameters' change from baseline.
^{c}SE of LS mean difference = SE of the LS mean difference.
*p < 0.05.
**p < 0.01.

### SUMMARY

In patients with HFrEF (mean age 60 years, 25% women, ischaemic heart disease 48%, mean LV ejection fraction 32%), Compound I (at plasma concentrations ≥2000 ng/mL) decreased LA minimal volume index (up to −2.4 mL/m2, p<0.01) and increased LA function index (up to 6.1, p<0.01), when compared with placebo. These results are consistent with pre-clinical findings of direct activation of LA contractility (see Examples 1 and 2).

Cardiac myosin activators enhance myofibrillar ATPase activity, leading to Ca²⁺-independent increases in both myocardial contractility and the duration of systole (i.e. SET) (Teerlink, Heart Fail Rev. (2009) 14(4):289-98), all features shared by Compound I and now supported by both preclinical and clinical observations. However, Compound I is also a selective and direct activator of cardiac actomyosin which does not hinder the maximal force production of the ventricular myocardium (Kampourakis et al., J Physiol (2018) 596(1):31-46; Nagy et al., Br J Pharmacol. (2015) 172(18):4506-18; Woody et al., Nat Commun. (2018) 9(1):3838). Moreover, Compound I directly increases force production in LA fibers, known to consist of intrinsically weaker (alpha) myosin motors (Aksel et al., Cell Rep. (2015) 11(6):910-20), further highlighting its ability to preserve/enhance myosin's intrinsic power generation (power stroke).

These studies confirm that Compound I improved atrial dimension/function in patients with HFrEF.

### Example 6: Randomized, Double-Blind, Parallel-Group Study of Clinical Efficacy and Safety of Chronic Compound I Treatment in Patients with Reduced LVEF and Paroxysmal or Persistent AF

This example describes a design for a study intended to establish the clinical efficacy and safety of chronic treatment with Compound I in patients with reduced LVEF (<50%) and paroxysmal or persistent AF.

Primary efficacy objectives of the study will include evaluating the effects of Compound I on LV and LA volume and function as measured by TTE, as well as evaluating the clinical efficacy of Compound I on AF burden, measured continuously via implanted device or ILR.

Primary safety objectives of the study will include evaluating the clinical safety and tolerability of chronic treatment with Compound I.

Secondary objectives of the study will include:
Evaluating the effects of Compound I on other TTE parameters (e.g., SET, diastolic function);
Evaluating the effect of Compound I on biomarkers (e.g., NT-proBNP, high-sensitivity troponins);
Evaluating the clinical efficacy of Compound I on AF recurrence;
Evaluating the clinical efficacy of Compound I on NYHA;
Evaluating the clinical efficacy of Compound I on patient reported outcomes (e.g., KCCQ, AFEQT);
Evaluating the PK of Compound I after chronic treatment; and
Evaluating the PK-PD effects of Compound I.

Exploratory objectives of the study will include:
Evaluating the clinical efficacy of Compound I on AF burden, measured via Zio patch (all patients);
Evaluating the clinical efficacy of Compound I on days alive and out-of-hospital;
Evaluating the clinical efficacy of Compound I on outcomes (e.g., CV death, CV hospitalization, urgent HF or AF visits);
Evaluating the clinical efficacy of Compound I on the 6 minute walk test (6MWT); and
Evaluating the effect of Compound I on activity level (e.g., accelerometry).

### Study Design

Two cohorts (Cohort 1 and Cohort 2) will be enrolled. Enrollment of up to a total of approximately 200 subjects is planned; however, additional cohorts may be enrolled. Of the 200 patients, 100 will have an implantable device or ILR (Cohort 1) and 100 will be in Cohort 2. The expected study duration for an individual patient is up to 8 months, including about 2-6 weeks for screening, 6 months (24 weeks) for treatment, and 4 weeks for follow-up.

Each cohort will encompass four parallel groups of 25 patients each, receiving placebo, Compound I at 25 mg BID, Compound I at 50 mg BID, or Compound I at 75 m BID.

### Inclusion Criteria

This study is to be performed in patients who meet the following criteria:

1. Men or women 18 to 85 years of age at the Screening visit
2. Documented reduced LVEF (<50%), based on most recent TTE performed within past 12 months or screening echo.
The most recent qualifying LVEF must not have been performed during an AF episode, and, if applicable, must have been performed at least 30 days after any of the following:
   1) hospitalization for an event likely to decrease EF (e.g., acute coronary syndrome/myocardial infarction, sepsis);
   2) an intervention likely to increase EF (e.g., cardiac resynchronization therapy, coronary revascularization); or
   3) the first ever presentation for HF
If diagnosis of HFrEF with LVEF≤40%, patient should be treated with GDMT (i.e., standard of care) including at least one of the following, unless not tolerated or contraindicated: beta-blocker, angiotensin converting enzyme (ACE) inhibitor, angiotensin receptor blocker (ARB), and angiotensin receptor neprilysin inhibitor (ARNI). Such therapies should have been given at stable doses for ≥3 weeks prior to randomization with no plan to modify during the study.
   4. NT-proBNP at screening ≥150 pg/mL (or ≥100 pg/mL if high BMI or Black patient)
   5. Diagnosis of atrial fibrillation (AF) as defined below:
For Cohort 1 (implanted device/ILR and paroxysmal AF), patient must meet all of the following criteria:
   AF burden can be continuously measured, i.e., patient has an implanted device with an atrial lead (pace-maker, ICD, CRT), or an implantable loop recorder (ILR) at screening or is willing to be implanted with ILR during screening period; AND
   AF burden at screening (based on device interrogation at screening) ranges from 2 to 70%. For patients willing to be implanted with ILR, qualifying AF burden will be based on a 2w Zio patch performed during screening. An ILR should be implanted only after patient is deemed eligible.
NOTE: device/ILR interrogation at screening should cover ≥2 continuous weeks
For Cohort 2 (no implanted device/ILR, paroxysmal or persistent AF), patient must meet all of the following criteria:
   AF burden cannot be continuously measured, AND
   patient has had a clinical diagnosis of AF (based on electrocardiographic evidence), not due to transient conditions (e.g., post-operative, etc.), AND
   patient has had at least one episode of sustained AF within 6 months prior to screening (based on medical records, or 12-lead ECG, or an episode of AF>10 minutes on Holter or patch, or prior ECV) and without evidence of long-standing persistent or permanent AF.
      6. For Cohort 1 patients only:
implanted device with an atrial lead/ILR must have remote data transmission capability
patient willing and able to transmit device data from home.

### Exclusion Criteria

Patients who meet any of the following criteria will be excluded from the study:

Related to AF:
Patient is considered for Cohort 1 (i.e., has implanted device or ILR, or willing to get an ILR) and has an AF burden at screening <2% or >70%
AF has reversible etiology (thyroid disease, alcohol, pulmonary embolism, early post-operative, acute pericarditis, trauma, etc.)
Patient with pulmonary hypertension treated with pulmonary vasodilators (endothelin receptor antagonists, PDES inhibitor, etc.)
Known channelopathy (e.g., long QT syndrome, Brugada syndrome, or CPVT)
AF diagnosed more than 10 years prior to screening
Evidence of long-standing persistent or permanent AF
AF episode during screening requiring an ECV or a change in antiarrhythmic therapy (NOTE: one rescreening allowed)
AF at randomization (12-lead ECG)
NOTE: patient may be randomized a few days later, after return to sinus rhythm
LA diameter (based on most recent TTE)>60 mm
Recent (<6 months prior to screening) or planned or likelihood of catheter ablation during the study
Recent (<1 month prior to screening) or planned introduction of new antiarrhythmic therapy during the study—no intent to change antiarrhythmic drug regimen
Electrical cardioversion (ECV) performed <1 month prior to screening, or during screening
(optional) Patient is unable to use and record 6-lead ECG at home.

Related to HF:
Inadequate echocardiographic acoustic windows
Class IV NYHA at screening
At screening, symptomatic hypotension or systolic blood pressure (BP)<90 mmHg, or diastolic blood pressure >95 mmHg
Severe aortic valvular disease or mitral stenosis, planned or anticipated mitral clip or mitral valve repair during the study, hypertrophic or infiltrative cardiomyopathy (e.g. amyloidosis), active myocarditis, constrictive pericarditis, or clinically significant congenital heart disease
Recent (≤90 days prior to screening) significant cardiovascular event (e.g., acute coronary syndrome, stroke, etc.)
Recent (≤90 days prior to screening) or planned cardiovascular intervention (including but not limited to: CABG, PCI, valvular repair)
Recent (≤45 days prior to screening) or planned device implantation, other than ILR implantation during screening (e.g., pacemaker, CRT)
Recent (≤90 days) hospitalization for heart failure or treatment with IV inotropes
End-stage HF defined as requiring left ventricular assist devices, intra-aortic balloon pump (IABP), or any type of mechanical support or awaiting heart transplantation.

Other exclusions:
Hypersensitivity to Formula I or any of the components of the Formula I formulation
Active infection, indicated clinically as determined by the investigator
History of malignancy of any type within 5 years prior to Screening, with the exception of the following surgically excised cancers occurring more than 2 years prior to Screening: in situ cervical cancer, nonmelanomatous skin cancers, ductal carcinoma in situ, and non-metastatic prostate cancer
Laboratory parameters:
   Severe renal insufficiency (defined as current estimated glomerular filtration rate [eGFR]<30 mL/min/1.73 m2 by simplified Modification of Diet in Renal Disease equation [sMDRD]).
   Serum potassium <3.5 or >5.5 mEq/L on most recent determination prior to randomization (1 repeat lab allowed)
   AST or ALT>3×ULN or Total bilirubin >2×ULN on most recent determination prior to randomization (1 repeat lab allowed)
   Any persistent (2 or more) out-of-range safety laboratory parameters (chemistry, hematology), considered by the investigator and medical monitor to be clinically significant.
   History or evidence of any other clinically significant disorder, condition, or disease (including substance abuse) that, in the opinion of the investigator or physician would pose a risk to subject safety or interfere with the study evaluation, procedures, completion, or lead to premature withdrawal from the study
   Life expectancy <6 months.
   Participated in a clinical trial in which the subject received any investigational drug (or is currently using an investigational device) within 30 days prior to Screening, or at least 5 times the respective elimination half-life (whichever is longer).

What is claimed is:

1. A method of treating atrial dysfunction in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 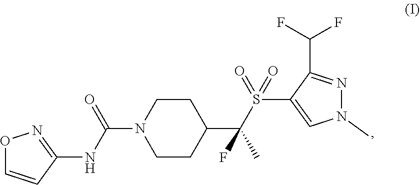 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the patient exhibits atrial fibrillation.

2. A method of treating atrial cardiomyopathy in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 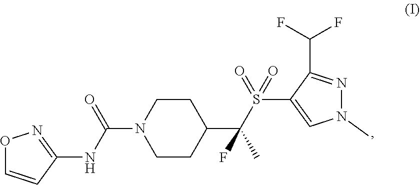 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the patient exhibits atrial fibrillation.

3. A method of treating atrial tachyarrhythmia in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 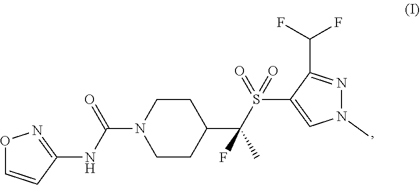 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the patient exhibits atrial fibrillation.

4. A method of treating atrial fibrillation in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 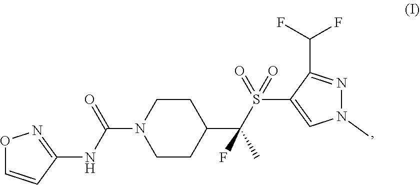 [SMILES: Cn1cc(S(=O)(=O)[C@@](C)(F)C2CCN(C(=O)Nc3ccon3)CC2)c(C(F)F)n1]
or a pharmaceutically acceptable salt thereof.

5. A method of reducing atrial fibrillation recurrence in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 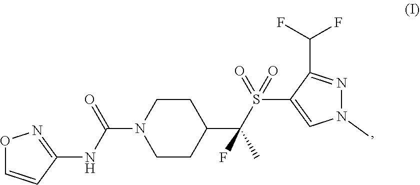 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein atrial fibrillation recurrence is reduced by 10% or greater.

6. A method of reducing atrial fibrillation burden in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl) fluoroethyl)-N-(isoxazol-3-yl)piperidine-1-carboxamide, having the structural formula (I) 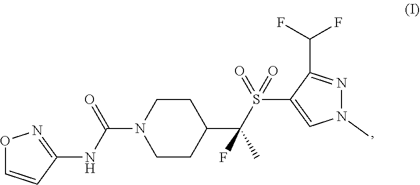 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein atrial fibrillation burden is reduced by 10% or greater.

7. A method of reducing the duration of an atrial fibrillation episode in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 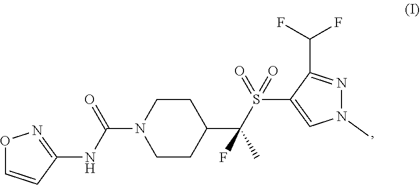 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the duration of the episode is reduced by 10% or greater.

8. A method of reducing the number of atrial fibrillation episodes during a monitoring period in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 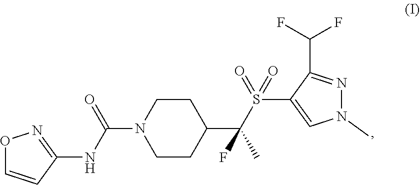 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the number of atrial fibrillation episodes is reduced by 10% or greater.

9. A method of maintaining sinus rhythm in a patient in need thereof, comprising administering to the patient a therapeutically effective amount of Compound I, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 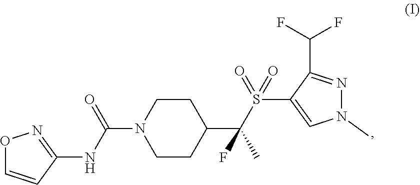 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the patient has sustained atrial tachyarrhythmia for 12 months or less prior to the administering step, further optionally wherein the atrial tachyarrhythmia is atrial fibrillation.

10. A method of restoring sinus rhythm in a patient exhibiting atrial tachyarrhythmia, comprising administering to the patient a therapeutically effective amount of Compound I in combination with cardioversion, wherein Compound I is (R)-4-(1-((3-(difluoromethyl)-1-methyl-1H-pyrazol-4-yl)sulfonyl)-1-fluoroethyl)-N-(isoxazol-3-yl)piperidine-1-carboxamide, having the structural formula (I) 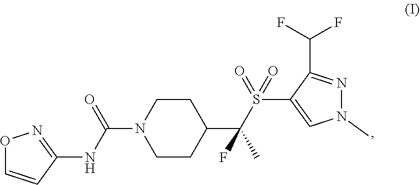 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the cardioversion is electrical cardioversion and further optionally wherein the atrial tachyarrhythmia is atrial fibrillation.

11. The method of any one of claims 1-10, wherein the patient also exhibits systolic dysfunction.

12. The method of claim 11, wherein the systolic dysfunction is a syndrome or disorder selected from the group consisting of heart failure, cardiomyopathy, cardiogenic shock, a condition that benefits from inotropic support after cardiac surgery, myocarditis, atherosclerosis, secondary aldosteronism, myocardial infarction, valve disease, systemic hypertension, pulmonary hypertension or pulmonary arterial hypertension, detrimental vascular remodeling, pulmonary edema, and respiratory failure; and optionally wherein
the heart failure is selected from heart failure with reduced ejection fraction (HFrEF), heart failure with preserved ejection fraction (HFpEF), congestive heart failure, and diastolic heart failure (with diminished systolic reserve),
the cardiomyopathy is selected from ischemic cardiomyopathy, dilated cardiomyopathy, atrial myopathy, left atrial myopathy, advanced hypertrophic cardiomyopathy, post-infarction cardiomyopathy, viral cardiomyopathy, toxic cardiomyopathy (optionally post-anthracycline anticancer therapy), metabolic cardiomyopathy (optionally cardiomyopathy in conjunction with enzyme replacement therapy), infiltrative cardiomyopathy (optionally amyloidosis), and diabetic cardiomyopathy,
the condition that benefits from inotropic support after cardiac surgery is ventricular dysfunction due to on-bypass cardiovascular surgery,
the myocarditis is viral myocarditis, and/or
the valve disease is mitral regurgitation or aortic stenosis.

13. The method of claim 11, wherein the systolic dysfunction is reduced left ventricular ejection fraction (LVEF).

14. The method of any one of claims 1-13, wherein the patient also exhibits diastolic dysfunction.

15. The method of any one of claims 1-14, wherein the patient has heart failure and a diagnosis of any one of NYHA Class II-IV.

16. The method of any one of claims 1-15, wherein the patient has HFrEF.

17. The method of claim 16, wherein the patient exhibits atrial fibrillation; and the therapeutically effective amount of Compound I alleviates one or more symptoms of HFrEF, maintains sinus rhythm, reduces atrial fibrillation recurrence, and/or prevents incident atrial fibrillation in the patient.

18. A method of preventing tachycardia-induced cardiomyopathy in a patient exhibiting atrial fibrillation, comprising administering to the patient a therapeutically effective amount of Compound I, wherein Compound I is (R)-4-(1-((3-(difluoromethyl)-1-methyl-1H-pyrazol-4-yl))sulfonyl)-1-fluoroethyl)-N-(isoxazol-3-yl)piperidine-1-carboxamide, having the structural formula (I) 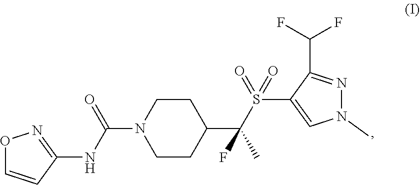 [SMILES: Cn1cc(S(=O)(=O)[C@@](C)(F)C2CCN(C(=O)Nc3ccon3)CC2)c(C(F)F)n1]
or a pharmaceutically acceptable salt thereof, optionally wherein the tachycardia-induced cardiomyopathy is heart failure, optionally heart failure with reduced ejection fraction (HFrEF).

19. The method of any one of claims 1-18 wherein the patient has sustained the tachyarrhythmia or atrial fibrillation for a duration of 12 months or less prior to the administering step.

20. The method of any one of claims 1-19, wherein the atrial fibrillation is paroxysmal or persistent, optionally wherein the atrial fibrillation is persistent and has been sustained for 12 months or less.

21. The method of any one of claims 1-20, wherein the patient has an atrial fibrillation burden of 2-70%.

22. The method of any one of claims 1-21, wherein the patient has postoperative AF.

23. The method of any one of claims 16-22, wherein the patient has a left ventricular ejection fraction (LVEF) of less than 50%, optionally wherein the patient has an LVEF 49% or less, 45% or less, 40% or less, 39% or less, 35% or less, 30% or less, 15-35%, 15-40%, 15-49%, 15-50%, 20-45%, 35-49%, 35-50%, 40-49%, 41-49%, 40-50%, or 41-50%.

24. The method of any one of claims 1-15 and 18-22, wherein the patient has a left ventricular ejection fraction (LVEF) of less than 60%.

25. The method of claim 24, wherein the patient has HFpEF.

26. The method of any one of claims 1-25, wherein the patient has left atrial enlargement.

27. The method of any one of claims 1-26, wherein the patient has atrial myopathy.

28. The method of claim 27, wherein the atrial myopathy is left atrial myopathy.

29. The method of any one of claims 1-28, wherein the patient has previously been treated with ablation or cardioversion.

30. The method of claim 29, wherein the ablation is catheter ablation.

31. The method of claim 29, wherein the cardioversion is electrical cardioversion.

32. The method of any one of claims 1-31, wherein the patient does not have any one or combination of the following:
a) AF burden of <2% or >70%;
b) AF with a reversible etiology;
c) pulmonary hypertension treated with pulmonary vasodilators, wherein the vasodilators are optionally endothelin receptor antagonists or PDES inhibitors;
d) known channelopathy, wherein the channelopathy is optionally long QT syndrome, Brugada syndrome, or CPVT;
e) AF diagnosed more than 10 years prior to the start of treatment;
f) long-standing persistent or permanent atrial fibrillation;
g) LA diameter >60 mm;
h) catheter ablation within <6 months prior to the start of treatment, or planned or likely catheter ablation during treatment;
i) introduction of new antiarrhythmic therapy <1 month prior to the start of treatment, or planned introduction of new antiarrhythmic therapy during treatment;
j) electrical cardioversion performed <1 month prior to the start of treatment;
k) heart failure of NYHA Class IV;
l) symptomatic hypotension, or systolic blood pressure <90 mmHg, or diastolic blood pressure >95 mmHg;
m) severe aortic valvular disease or mitral stenosis, planned or anticipated mitral valve repair during treatment, hypertrophic or infiltrative cardiomyopathy, active myocarditis, constrictive pericarditis, or clinically significant congenital heart disease;
n) significant cardiovascular event within ≤90 days prior to the start of treatment, wherein the cardiovascular event is optionally acute coronary syndrome or stroke;
o) cardiovascular intervention within ≤90 days prior to the start of treatment, wherein the cardiovascular intervention is optionally CABG, PCI, or valvular repair;
p) device implantation within ≤45 days prior to the start of treatment, wherein the device is optionally a pacemaker or CRT;
q) hospitalization for heart failure or treatment with IV inotropes within ≤90 days prior to the start of treatment;
r) end stage heart failure; or
s) life expectancy <6 months.

33. The method of any one of claims 1-32, wherein the patient is administered Compound I at a total daily dose of 10-350 mg.

34. The method of any one of claims 1-32, wherein the patient is administered Compound I at 10-175 mg BID, 25-325 mg QD, or 25-350 mg QD.

35. The method of any one of claims 1-32, wherein the patient is administered Compound I at 10-75 mg BID, optionally at 10, 25, 50, or 75 mg BID.

36. The method of any one of claims 1-35, wherein the Compound I is administered to the patient orally.

37. The method of any one of claims 1-36, wherein Compound 1 is administered in a dose resulting in Compound I plasma concentrations of 1000 to 8000 ng/mL in the patient, optionally wherein the dose results in Compound I plasma concentrations of <2000 ng/mL, 2000-3500 ng/mL, or >3500 ng/mL in the patient.

38. The method of any one of claims 1-37, wherein Compound I is ingested by the patient with food or within about two hours, within about one hour, or within about 30 minutes of food.

39. The method of any one of claims 1-38, wherein Compound I is provided in a solid form with a mean particle size greater than 15 μm in diameter, less than 10 μm in diameter, between 15 μm and 25 μm in diameter, between 1 μm and 10 μm in diameter, or between 1 μm and 5 μm in diameter.

40. The method of any one of claims 1-39, wherein the patient has undergone an electrical cardioversion before or after the administering step, optionally where the electrical cardioversion is performed no more than 24 hours before or after the administering step.

41. The method of any one of claims 1-40, further comprising administering to the patient an additional medication for improving cardiovascular conditions in the patient, optionally wherein the additional medication is a beta blocker, an anticoagulant, a vitamin K antagonist, a calcium channel blocker, a diuretic, an angiotensin-converting enzyme (ACE) inhibitor, an angiotensin II receptor blocker (ARB), a mineralocorticoid receptor antagonist, an angiotensin receptor-neprilysin inhibitor (ARNI), an SGLT2 inhibitor, an sGC activator or modulator, an antiarrhythmic medication, or any combination thereof.

42. The method of any one of claims 1-40, further comprising administering to the patient an anticoagulant and an antiarrhythmic agent.

43. The method of any one of claims 1-40, further comprising administering to the patient an anticoagulant and a rate control agent, optionally wherein the rate control agent is a beta-blocker, digoxin, or amiodarone.

44. The method of any one of claims 1-40, further comprising administering to the patient an anticoagulant; a diuretic; and an angiotensin-converting enzyme (ACE) inhibitor, angiotensin II receptor blocker (ARB), and/or mineralocorticoid receptor antagonist.

45. The method of any one of claims 1-44, wherein the method results in any one or combination of the following:
a) reduced risk of urgent outpatient intervention for atrial dysfunction, systolic dysfunction, or both;
b) improved quality of life as measured through 6-MWT or KCCQ;
c) improved exercise capacity;
d) improvement in a patient's NYHA classification;
e) delay in clinical worsening;
f) reduction in severity of cardiovascular-related symptoms;
g) increased left atrial ejection fraction (LAEF);
h) decreased left atrial volume (LAₘᵢₙVI); and
i) improved left atrial function index (LAFI).

46. The method of any one of claims 1-45, wherein the method results in reduced cardiovascular death or hospitalization.

47. Compound I, or a pharmaceutical composition comprising Compound I and a pharmaceutically acceptable excipient, for use in the method of any one of claims 1-46.

48. Compound I for use in the manufacture of a medicament for treating a patient in the method of any one of claims 1-46.
